(19) United States
(12) Patent Application Publication
(10) Pub. No.: US 2015/0153364 A1
(43) Pub. Date: Jun. 4, 2015

(54) BIOMARKERS FOR TANGLE-PREDOMINANT DEMENTIA

(71) Applicant: The Trustees of Columbia University in the City of New York, New York, NY (US)

(72) Inventor: John F. Crary, Brooklyn, NY (US)

(73) Assignee: The Trustees of Columbia University in the City of New York, New York, NY (US)

(21) Appl. No.: 14/409,108

(22) PCT Filed: Jun. 21, 2013
(86) PCT No.: PCT/US2013/047047

§ 371 Date: Dec. 18, 2014

Related U.S. Application Data
(60) Provisional application No. 61/662,644, filed on Jun. 21, 2012.

(51) Int. Cl. G01N 33/68; C12Q 1/68

(52) U.S. Cl. CPC G01N 33/6896; C12Q 1/6883; C12Q 2600/156; G01N 2333/4709; G01N 2800/2814

(57) ABSTRACT

This invention relates to the field of screening for, identifying, and diagnosing tangle-predominant dementia (TPD). Specifically, this invention provides various biomarkers for this disease, and methods of using these biomarkers to correctly subclassify patients with Alzheimer's-type dementia by differentiating TPD patients from those with classical Alzheimer's disease (AD), as well as prodromal AD, and mild cognitive impairment due to AD.

### CROSS REFERENCE TO RELATED APPLICATION

The present application claims priority to U.S. patent application Ser. No. 61/662,644 filed Jun. 21, 2012, which is hereby incorporated by reference in its entirety.

### FIELD OF INVENTION

This invention relates to the field of screening for, identifying, and diagnosing tangle-predominant dementia (TPD), also known as tangle-only dementia, tangle-predominant senile dementia, senile dementia of the tangle type, senile dementia with tangles, limbic neurofibrillary tangle dementia, Alzheimer's disease with tangles only, as well as other names. Specifically, this invention provides various biomarkers for this disease, and methods of using these biomarkers to correctly subclassify patients with Alzheimer's-type dementia by differentiating TPD patients from those with classical Alzheimer's disease (AD), as well as prodromal AD, and mild cognitive impairment due to AD.

### BACKGROUND OF THE INVENTION

The presence of abnormal neuronal and glial filamentous inclusions composed of the microtubule-associated protein tau defines a heterogeneous group of neurodegenerative disorders, termed tauopathies (Dickson (2009)). Alzheimer's disease (AD) is currently classified as a secondary tauopathy, with tangles thought to arise as a result of increased levels of toxic species of the amyloid-beta peptide (Aβ) (Hardy (2006)). Classically, Alzheimer's disease is associated with amyloid plaques and neurofibrillary tangles (NFT). However, the relationship between amyloid plaques and neurofibrillary degeneration in AD remains unclear. Furthermore, clinicopathological studies do not reveal a strong correlation between amyloid plaques and cognitive impairment (Dickson et al. (1992); Katzman et al. (1998); Terry et al. (1991)).

The discovery of mutations in the microtubule-associated protein tau gene (MAPT) that cause frontotemporal lobar degeneration (FTLD) show that tau dysfunction on its own is sufficient to induce neurodegeneration (Hutton et al. (1998); Gasparini et al. (2007)), but the majority of patients with tangles lack such mutations. One example of a sporadic non-mutational primary tauopathy is tangle-predominant dementia (TPD) or tangle-only dementia (TOD) (Bancher and Jellinger (1994); Nelson et al. (2009); Ulrich et al. (1992)). These patients develop neurofibrillary tangles that are regionally, morphologically, ultrastructurally and biochemically identical to those in moderate-stage AD, yet lack significant Aβ deposition as plaques (Santa-Maria et al. (2012)).

TPD patients exhibit neurofibrillary tangles in the medial temporal lobe, progressing to a regional distribution corresponding to moderate-stage AD (Braak stages III-IV) (Braak and Braak (1991)). However, the severity of NFT in TPD more closely resembles end-stage disease (Noda et al. (2006)). Currently, neither clinical features nor diagnostic tests can differentiate TPD from early to moderate AD, and the only way to diagnose TPD is post-mortem.

The etiology of TPD is not known. Authors have grouped TPD with frontotemporal lobar degeneration (FTLD) with MAPT mutation, but this classification is imperfect given the differences in symptomatology and neuropathological features of FTLD and TPD (Cairns et al. (2007); Ikeda et al. (1999)).

The scarcity of Aβ deposition that differentiates TPD from classical “plaque and tangle” AD is striking with rigorous histological analysis failing to find more than scattered diffuse plaques or vascular amyloid in a small minority of patients (Ulrich et al. (1992); Jellinger and Attems (2011); Bancher and Jellinger (1994)). Tauopathies are classified neuropathologically using the distribution, morphology and ultrastructure of neurofibrillary tangles, yet no features can differentiate the NFTs in TPD from those in moderate-stage AD. The prevalence of neurofibrillary tangles in normal elderly individuals has prompted suggestions that TPD is a form of pathological or “accelerated” aging (Bouras et al. (1994); Junn and Mouradian (2012); Price and Morris (1999); Savva et al. (2009)). Finally, TPD may be an AD variant (Jellinger and Bancher (1998)).

The mechanism of how NFTs form in the absence of amyloid plaques is of critical importance. The amyloid cascade hypothesis posits that increased Aβ is the disease trigger in AD, leading to NFT formation and neurodegeneration (Hardy and Selkoe (2002)). The toxic species in AD may be soluble Aβ in the form of pre-fibrillar diffusible assemblies, rather than Aβ deposited in plaques (Walsh and Selkoe (2007)). While the possibility that APP or its catabolites contribute to TPD has not previously been tested experimentally, the consensus criteria for the neuropathological diagnosis of AD require the presence of insoluble Aβ deposited in plaques together with NFT (Hyman et al. (2012); Montine et al. (2012)).

Currently, there is no way to clinically differentiate TPD from classical plaque and tangle AD, yet the distinction is critical for implementing Aβ-targeted therapies. Given the low Aβ levels in TOD, it is unlikely that Aβ-targeted agents will be useful and they may subject TPD patients to unnecessary risk. Given the predominantly amnestic symptoms, TPD patients may be clinically classified as having mild cognitive impairment due to AD (Albert et al. (2011)). Unfortunately, the cerebrospinal fluid biomarkers for AD (i.e., low Aβ and high tau) are predicted to be positive in TPD (Trojanowski et al. (2010)). The exception is positron emission tomography (PET)-based amyloid imaging, which may increase recognition of TPD. Notably, as many as one third of amnestic mild cognitive impairment patients are ¹¹C-PIB negative (Devanand et al. (2010)), which is compatible with the findings here and by others that TPD is more widespread than acknowledged by the research community. Improved methodology to identify and treat TPD would be highly clinically useful.

Thus, there is a need for methods of screening for, diagnosing or identifying tangle-only or tangle-predominant dementia, particularly differentiating TPD from amyloid driven neurodegeneration (i.e., classical Alzheimer's disease) and other dementing illnesses.

### SUMMARY OF THE INVENTION

This invention is based on the surprising discovery that TPD patients, who develop Alzheimer's-type neurofibrillary tangles that are biochemically identical to those in early to moderate-stage AD, have very low levels of soluble Aβ. Furthermore, it was discovered that there is non-amyloidogenic APP processing in TPD brain. Genetic analysis demonstrated that TPD is associated with the MAPT H1 haplotype in the absence of a coding region mutation, and an additional significant association between TPD and a genomic variation in the MAPT 3′ UTR, suggesting a novel mechanism whereby post-transcriptional regulation of MAPT contributes to tauopathy. These various novel biomarkers can be used to screen for, diagnose, and/or identify TPD in patients who exhibit cognitive impairment, and in particular, to differentiate the disease from AD. Additionally, the knowledge that the variation in the MAPT 3′UTR is associated with tauopathy reveals new methods of cellular drug screens.

One embodiment of the present invention is a method for screening, diagnosing, predicting and/or identifying TPD, comprising identifying a subject with cognitive impairment, obtaining biological tissue and/or bodily fluid from the subject, purifying and/or isolating protein from said biological tissue and/or bodily fluid, and detecting the levels of Aβ protein or peptide in the purified and/or isolated protein sample. The level of Aβ is compared to the levels in a protein sample from a healthy control and/or a patient known to have AD. If the levels of Aβ are different, either qualitatively, e.g., by visualization, or quantitatively, e.g., comparison to a known quantity of the protein in a healthy control and/or a subject with AD, the patient can be determined, diagnosed, predicted or identified as having TPD. Specifically, if the level of Aβ in the protein sample from the subject is decreased or lower than the level of Aβ in the protein sample from the healthy control and/or the subject known to have AD, then the patient can be diagnosed or identified as having TPD.

In a preferred embodiment, the patient who is being tested has a cognitive impairment that might be diagnosed as Alzheimer's disease.

The purified and/or isolated protein sample can be obtained from any biological tissue. Preferred biological tissues include, but are not limited to, brain, epidermal, whole blood, and plasma.

The purified and/or isolated protein sample can be obtained from any bodily fluid. Preferred biological fluids include, but are not limited to, cerebrospinal fluid, plasma, saliva, sweat, and urine.

The protein can be obtained and processed from the biological tissue or bodily fluid by any method known in the art, in order to obtain a purified and/or isolated protein sample.

Detection of the level of Aβ can be accomplished by any method known in the art, including methods which result in qualitative results, such as ones where the existence of the protein can be visualized, either by the naked eye or by other means, and/or quantitative results. Such methods would include, but are not limited to, quantitative Western blots, immunoblots, quantitative mass spectrometry, enzyme-linked immunosorbent assays (ELISAs), radioimmunoassays (RIA), immunoradiometric assays (TRIVIA), and immunoenzymatic assays (TEMA) and sandwich assays using monoclonal and polyclonal antibodies.

In a preferred embodiment, the results of these methods in the subject are compared to the results of the same method in a healthy control and/or a subject known to have AD.

In a preferred embodiment, the quantity of Aβ is measured in the protein sample from the subject and compared to a reference value of the quantity of Aβ in a healthy control and/or a subject with Alzheimer's disease, wherein the reference value represents a known diagnosis or prediction of AD or normal cognitive function, and finding a deviation in the quantity of Aβ from protein sample of the subject and the reference value, wherein if the quantity of Aβ from protein sample of the subject is decreased or lower than the reference value, then the subject can be determined, diagnosed, predicted or identified as having TPD.

In a preferred embodiment, the level of Aβ42 is detected and/or measured. In another preferred embodiment, the level of 1 Aβ40 is detected and/or measured, and in a most preferred embodiment, both the level of Aβ42 and the level of Aβ40 are detected and/or measured.

A further embodiment of the present invention is a method for screening, diagnosing, predicting and/or identifying TPD, comprising obtaining biological tissue and/or bodily fluid from a subject, purifying and/or isolating protein from said biological tissue and/or bodily fluid, and detecting the levels of sAPPα and/or sAPPβ in the purified and/or isolated protein sample. These levels of sAPPα and/or sAPPβ are compared to the levels in a protein sample from a healthy control. If the levels of sAPPα and/or sAPPβ are different, either qualitatively, e.g., by visualization, or quantitatively, e.g., comparison to a known quantity of the proteins in a healthy control, the subject can be diagnosed or identified as having TPD. Specifically, if the level of sAPPα in the protein sample from the subject is increased or higher than the level of sAPPα in the protein sample of the healthy control, then the subject can be diagnosed or identified as having TPD. If the level of sAPPβ in the protein sample from the subject is decreased or lower than the level of sAPPβ in the protein sample from the healthy control, then the subject can be diagnosed or identified as having TPD. While one or the other protein can be tested for, a preferred embodiment is to test for both proteins in the isolated and/or purified protein sample from the subject.

In a preferred embodiment, the subject who is being tested has a cognitive impairment that might be diagnosed as Alzheimer's disease.

The purified and/or isolated protein sample can be obtained from any biological tissue. Preferred biological tissues include, but are not limited to, brain, epidermal, whole blood, and plasma.

The purified and/or isolated protein sample can be obtained from any bodily fluid. Preferred bodily fluids include, but are not limited to, cerebrospinal fluid, plasma, saliva, sweat, and urine.

The protein can be obtained and processed from the biological tissue or bodily fluid by any method known in the art, in order to obtain a purified and isolated protein sample.

Detection of the levels of sAPPα and/or sAPPβ can be accomplished by any method known in the art, including methods which result in qualitative results, such as ones where the existence of the protein can be visualized, either by the naked eye or by other means, and/or quantitative results. Such methods would include, but are not limited to, quantitative Western blots, immunoblots, quantitative mass spectrometry, enzyme-linked immunosorbent assays (ELISAs), radioimmunoassays (RIA), immunoradiometric assays (IRMA), and immunoenzymatic assays (IEMA) and sandwich assays using monoclonal and polyclonal antibodies.

In a preferred embodiment, the results of these methods in the subject are compared to the results of the same method in a healthy control.

In a preferred embodiment, the quantity of sAPPα and/or sAPPβ is measured in the protein sample from the subject and compared to a reference value of the quantity of sAPPα and/or sAPPβ in a healthy control, wherein the reference value represents a known diagnosis or prediction of normal cognitive function, and finding a deviation in the quantity of sAPPα and/or sAPPβ from protein sample of the subject and the reference value, wherein if the quantity of sAPPα from protein sample of the subject is increased or higher than the reference value, then the subject can be determined, diagnosed, predicted or identified as having TPD, and sAPPβ from protein sample of the subject is decreased or lower than the reference value, then the subject can be determined, diagnosed, predicted or identified as having TPD

Apolipoprotein E (ApoE) alleles, which correlate with AD risk and amyloid plaque load, were found to have different frequencies in patients with TPD. Specifically, a decrease in the ApoE allele, ε4, was seen in patients with TPD as compared to AD, and an increase in alleles, ε2 and ε3, in patients with TPD as compared to patients with AD. Thus, a further embodiment of the present invention is a method for screening, diagnosing, predicting and/or identifying tangle-predominant dementia, comprising obtaining biological tissue and/or bodily fluid from a subject, purifying and/or isolating nucleic acid, including but not limited to cDNA and genomic DNA from the biological tissue and/or bodily fluid, and detecting the presence and/or absence of certain ApoE alleles, including ε2, ε3, and ε4. Specifically, the absence of the A allele in the patient would identify or diagnose the patient as having TPD, and the presence of the ε2 and/or ε3 alleles would identify or diagnose the subject as having TPD. While one of the alleles can be tested for, a preferred embodiment is to test for two, and a preferred embodiment is to test for all three.

In a preferred embodiment, the subject who is being tested has a cognitive impairment that might be diagnosed as Alzheimer's disease.

The purified and isolated nucleic acid can be obtained from any biological tissue. Preferred biological tissues include, but are not limited to, brain, epidermal, whole blood, and plasma.

The purified and isolated nucleic acid can be obtained from any bodily fluid. Preferred bodily fluids include, but are not limited to, cerebrospinal fluid, plasma, saliva, sweat, and urine.

The nucleic acid can be purified and isolated using any method known in the art.

Detection of the Apo E alleles can be accomplished by any method known in the art, including, but not limited to, sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis.

One preferred method for the detection of the Apo E alleles is to amplify and sequence the Apo E gene and determine if the alleles are present by a comparison to the known sequences for the alleles, ε2, ε3, and ε4.

Detection of the Apoe E alleles can also be accomplished by allele-specific PCR. In this method, primers specific for each Apo E allele are designed from the sequence of the Apo E gene. These primers will anneal to the purified and isolated nucleic acid of the patient only if the particular allele is present.

Another preferred embodiment of this method includes hybridizing the isolated and purified genomic DNA from Apo E allele from the subject with probes comprising the nucleotide sequence of the Apo E alleles. If the probes comprising the nucleotide sequence of Apo E alleles, ε2 and/or ε3 hybridize to the isolated and purified genomic DNA from the subject and/or the probes comprising the nucleotide sequence of ε4 does not hybridize to the isolated and purified genomic DNA from the subject, the subject is determined, diagnosed, predicted or identified as having TPD. In these embodiments, the isolated and purified genomic DNA or the probes must be labeled by methods known in the art for visualization if hybridization occurs.

A further embodiment of the present invention is based upon the surprising findings set forth herein that the haplotype H1 of the MAPT locus, and especially certain single nucleotide polymorphisms (SNP) in the 3′UTR, are highly associated with TPD.

Thus, this embodiment of the present invention is a method for screening, diagnosing and/or identifying tangle-predominant dementia, comprising obtaining biological tissue and/or bodily fluid from a subject, purifying and/or isolating nucleic acid, including, but not limited to, genomic DNA and RNA from the biological tissue and/or fluid, and detecting the presence of the H1 haplotype in the genomic DNA, wherein the presence of the H1 haplotype diagnoses or identifies the patient as having TPD.

In a preferred embodiment, the subject who is being tested has a cognitive impairment that might be diagnosed as Alzheimer's disease.

The purified and/or isolated nucleic acid can be obtained from any biological tissue. Preferred biological tissues include, but are not limited to, brain, epidermal, whole blood, and plasma.

The purified and isolated nucleic acid can be obtained from any bodily fluid. Preferred bodily fluids include, but are not limited to, cerebrospinal fluid, plasma, saliva, sweat, and urine.

The nucleic acid can be purified and isolated using any method known in the art.

Detection of the H1 haplotype can be accomplished by any method known in the art, including, but not limited to, sequencing, hybridization with probes including Southern blot analysis and dot blot analysis, polymerase chain reaction (PCR), PCR with melting curve analysis, PCR with mass spectrometry, fluorescent in situ hybridization, DNA microarrays, and single-strand conformation analysis.

One preferred method of detection of the H1 haplotype is to amplify the MAPT locus with primers, sequencing the MAPT locus and determining if the H1 haplotype is present by a comparison to known sequences of the H1 haplotype, such as those found in Table 9. Primers useful in this technique can be manufactured using the sequence of the MAPT locus as well as the alleles of the H1 haplotype listed in Table 9. The MAPT H1 and H2 haplotype can be determined by PCR using the Delln9 238 bp marker.

Detection of the H1 haplotype can also be accomplished by allele-specific PCR. In this method, primers specific for the H1 haplotype are designed from the sequence of the MAPT H1 haplotype. These primers will anneal to the purified and/or isolated genomic DNA of the patient only if the H1 haplotype is present.

Yet another embodiment of the present invention is a method for screening, diagnosing, predicting and/or identifying tangle-predominant dementia, comprising obtaining biological tissue and/or bodily fluid from a subject, purifying and/or isolating the genomic DNA from the tissue or fluid, and detecting the presence or absence of particular polymorphisms found in the 3′UTR of the MAPT gene.

Specifically the presence of the polymorphism designated rs35134565, with the nucleotide sequence set forth in SEQ ID NO: 1, and/or the absence of the polymorphism designated rs5820605, with the nucleotide sequence set forth in SEQ ID NO: 2, determines, diagnoses, predicts or identifies a patient as having TPD.

In a preferred embodiment, the subject who is being tested has a cognitive impairment that might be diagnosed as Alzheimer's disease.

The purified and/or isolated genomic DNA can be obtained from any biological tissue. Preferred biological tissues include, but are not limited to, brain, epidermal, whole blood, and plasma.

The purified and isolated genomic DNA can be obtained from any bodily fluid. Preferred bodily fluids include, but are not limited to, cerebrospinal fluid, plasma, saliva, sweat, and urine.

The genomic DNA can be purified and isolated using any method known in the art.

Detection of the polymorphisms can be accomplished by any method known in the art, including, but not limited to, sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis.

One preferred method of detection of the polymorphism is to amplify the MAPT 3′UTR with primers, sequencing the MAPT 3′UTR and determining if the polymorphisms are present or absent. In a preferred embodiment, the primers with the DNA sequences of SEQ ID NOs: 4 and 5 can be used. The nucleotide sequence obtained of the genomic DNA of the 3′UTR of the MAPT gene of the patient is compared to the nucleotide sequences of the polymorphisms. If the DNA from the patient contains the polymorphism designated rs35134565 with the nucleotide sequence of SEQ ID NO: 1 and/or does not contain the polymorphism designated rs5820605, with the nucleotide sequence set forth in SEQ ID NO: 2, the patient is diagnosed or identified as having TPD.

Another preferred embodiment of this method includes hybridizing the isolated and purified genomic DNA from the 3′UTR of the MAPT gene from the patient with probes comprising the nucleotide sequence of SEQ ID NOs: 1 and/or 2 and/or nucleotide sequence of the antisense strand of the SEQ ID NOs 1 and/or 2. If the probes comprising the nucleotide sequence of SEQ ID NO: 1 hybridizes to the isolated and purified genomic DNA from the 3′UTR of the MAPT gene from the subject and/or the probes comprising the nucleotide sequence of SEQ ID NO: 2 do not hybridize to the isolated and purified genomic DNA from the 3′UTR of the MAPT gene from the subject, the subject is diagnosed or identified as having TPD. In these embodiments, the isolated and purified genomic DNA or the probes must be labeled by methods known in the art for visualization if hybridization occurs.

Thus, there are a total of nine biomarkers that can be used to screen for, diagnose and/or identify tangle-predominant dementia or tangle-only dementia. These are:
1. a decreased level of Aβ;
2. an increased level of sAPPα;
3. a decreased level of sAPPβ;
4. the presence of the ApoE allele, ε2;
5. the presence of the ApoE allele, ε3;
6. the absence of the ApoE allele, ε4;
7. the presence of the H1 haplotype;
8. the presence of the polymorphism designated rs35134565 with the nucleotide sequence comprising SEQ ID NO: 1; and
9. the absence of the polymorphism designated rs5820605, with the nucleotide sequence comprising SEQ ID NO: 2.

It is contemplated by the present invention that a method that detects any one of these biomarkers can be performed and is sufficient to screen for, diagnose, predict and/or identify tangle-predominant dementia or tangle-only dementia. In a preferred embodiment, methods that detect at least two biomarkers are performed, in a more preferred embodiment, methods that detect at least three biomarkers are performed, in a more preferred embodiment, methods that detect at least four biomarkers are performed, in a more preferred embodiment, methods that detect at least five biomarkers are performed, in a more preferred embodiment, methods that detect at least six biomarkers are performed, in a more preferred embodiment, methods that detect at least seven biomarkers are performed, in a more preferred embodiment, methods that detect at least eight biomarkers are performed, and in the most preferred embodiment, methods that detect all nine biomarkers are performed.

In a further preferred embodiment, a method to detect the H1 haplotype is performed, and if the haplotype is found in the isolated and purified genomic DNA from the patient, the isolated and purified genomic DNA is then analyzed for the presence of the polymorphism designated rs35134565 with the nucleotide sequence of SEQ ID NO: 1; and the absence of the polymorphism designated rs5820605, with the nucleotide sequence set forth in SEQ ID NO: 2.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 are images of samples from patients with TPD, AD, and controls. FIG. 1a depicts immunohistochemical staining of the medial temporal lobe in a TPD patient, with p-tau specific AT8 antisera; FIG. 1b depicts immunohistochemical staining of the medial temporal lobe in an AD patient, with p-tau AT8 antisera; and FIG. 1c depicts immunohistochemical staining of the medial temporal lobe in a control patient, with p-tau AT8 antisera (scale bar, 7.5 mm). FIGS. 1d and 1e are P-tau immunohistochemical and Bielschowsky silver stains in a TPD patient (CA1), respectively (scale bar, 200 μM). FIGS. 1f and 1g are Bielschowsky silver stains in a TPD patient (scale bar, 50 μM). FIGS. 1h and 1i are immunohistochemical staining with 3R tau (FIG. 1h) and 4R tau (FIG. 1i) in the hippocampus of a TPD patient (scale bar, 50 μM). FIG. 1j is ultrastructural analysis of epoxy resin ultrathin sections from CA1 of a TPD patient. Arrowheads depict intracellular neurofibrillary tangles (scale bar, 5 μm). FIGS. 1k and 1l are high-power imaging of ultrathin sections from CA1 of a TPD patient and an AD patient, respectively (scale bar, 200 nm).

FIG. 2a is a representative immunoblot using antisera targeting total tau (HT7) and total protein samples from the frontal cortex (BA9) and hippocampus (CA1) demonstrating 69, 64 and 55 kD bands in TPD and control. A high-molecular weight of about 105 kD band is observed in TPD in CA1. FIG. 2b is a representative immunoblot using antisera to 3R and 4R tau show similar banding in TPD and controls. FIG. 2c are graphs showing the amount of 3R tau, 4R tau and the 3R/4R ratio in control and TPD patients in BA9. Comparisons are by Student's t-test, p<0.05, ** p<0.01, *** p<0.001. FIG. 2d is a representative immunoblot with antisera recognizing total tau (HT7) on sarkosyl insoluble tau fractions in AD, TPD and controls. FIG. 2e are images from ultrastructural examination of the sarkosyl-insoluble tau fractions in AD and TPD (scale bar, 200 nm).

FIGS. 3a through 3c show images from immunohistochemical staining with antisera targeting Aβ in the frontal cortex (BA9) reveals Aβ deposition in control and Alzheimer disease (AD), but not TPD (scale bar, 1 mm). FIGS. 3d through 3f are graphs of ELISA results of Aβ42 levels in BA9 and CA1 of control, TPD and AD (FIG. 3d); Aβ40 levels in BA9 and CA1 of control, TPD and AD (FIG. 3e); and the ratio of Aβ42/Aβ40 in BA9 and CA1 of control, TPD and AD (FIG. 3f). Comparisons are by one-way ANOVA and Tukey's test, * p<0.05, ** p<0.01, *** p<0.001.

FIG. 4a is a representative quantitative immunoblot of extracts from BA9 with APP, sAPPα, and sAPPβ, normalized to GAPDH. FIG. 4b is a graph quantifying the results of FIG. 4a. FIG. 4c is a graph of levels of the APP mRNA in TPD, AD and control. Comparisons are by Student's t-test (b) or one-way ANOVA/Tukey's test (c) or * p<0.05, ** p<0.01, *** p<0.001.

FIG. 5 shows linkage disequilibrium (LD) in MAPT. The genotypes for the MAPT haplotype tagging SNPs (rs1467967, rs242557, rs3785883, rs2471738, rs9468 and rs7521) together with rs5820605 and rs35134656 from cases and controls (n=82) were analyzed in Haploview v4.2 to generate the LD plot. The white horizontal bar represents the chromosomal distance between the polymorphisms. Pairwise D′ values are indicated within the diamonds. Variants with strong LD are shown in red. Those without LD are white and variants with uninformative data light blue.

FIG. 6 shows the two polymorphisms associated with TPD, rs35134656 (SEQ ID NO: 1) and rs5820685 (SEQ ID NO: 2).

FIG. 7 is a schematic of the dual luciferase reporter system used in Example 7.

FIG. 8a are graphs of the total tau mRNA, 3R tau mRNA, 4R tau mRNA, and the ratio of 3R/4R tau mRNA in control, TOD and AD as measured by QPCR. FIG. 8b is a graph of luciferase activity versus time in the cell transiently transfected with the three constructs designated, H1^{3′UTR-rs5820605}, H1^{3′UTR}, and H2^{3′UTR} and an empty vector. FIG. 8c is a graph of luciferase activity in the same cells after application of 300 μM Aβ.

### DETAILED DESCRIPTION OF THE INVENTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The terms “tangle-only dementia”, “tangle-predominant dementia”, “TOD”, and “TPD”, “tangle-predominant senile dementia”, “senile dementia of the tangle type”, “senile dementia with tangles”, and “limbic neurofibrillary tangle dementia”, will be used interchangeably in this application and are defined as patients who develop neurofibrillary tangles that are regionally, morphologically, ultrastructurally and biochemically similar to those in moderate-stage AD, but lack significant Aβ deposition as plaques (Santa-Maria et al. (2012)).

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments of the present invention, the “patient” is one suffering with cognitive impairment ranging from mild to severe, or with pre-dementia in the prodromal phase.

The terms “screen” and “screening” and the like as used herein means to test a subject or patient to determine if they have a particular illness or disease, in this case TPD.

The terms “diagnosis”, “diagnose”, diagnosing” and the like as used herein means to determine what physical disease or illness a subject or patient has, in this case TPD.

The terms “identification”, “identify”, “identifying” and the like as used herein means to recognize a disease in a subject or patient, in this case TPD.

The terms “prediction”, “predict”, “predicting” and the like as used herein means to tell in advance based upon special knowledge.

The term “reference value” as used herein means an amount of a quantity of a particular protein or nucleic acid in a sample from a healthy control or a subject known to have AD.

The term “healthy control” would be a human subject who is not suffering from dementing illness and has normal cognitive function. Moreover, it is preferred that the healthy control be age-matched to the subject, within a reasonable range.

The term “mild cognitive impairment” or “MCI” as used in this application means an intermediate stage between the expected cognitive decline of normal aging and the more serious decline of dementia. It is a brain function syndrome involving the onset and evolution of cognitive impairments beyond those expected based on the age and education of the individual, but which are not significant enough to interfere with their daily activities. It is often found to be a transitional stage between normal aging and dementia. Although MCI can present with a variety of symptoms, when memory loss is the predominant symptom it is termed “amnestic MCI” and is frequently seen as a prodromal stage of Alzheimer's disease.

The terms “Aβ” or “Abeta” are used interchangeably in this application and mean the amyloid beta protein or peptide, derived from the amyloid precursor protein (APP) (Thinakaren and Koo (2008)). Proteolysis of APP by α-secretase or β-secretase produces secreted N-terminal fragments termed sAPPα and sAPPβ respectively as well as C-terminal fragments (CTFs). Cleavage by γ-secretase of the β-CTF yields Aβ peptides of predominantly 40 or 42 amino acids. Aβ is the main component of amyloid plaques, deposits found in the brains of patients with Alzheimer's disease.

The terms “MAPT”, “MAPT gene” and “MAPT locus” are used interchangeably in this application and mean the microtubule-associated protein tau gene.

The terms “3′UTR” or “3′UTR of the MAPT gene” are used interchangeably in this application and mean the critical cis-acting regulatory elements that are capable of regulating gene expression on the post-transcriptional level by influencing mRNA stability and localization, among other functions (Aronov et al. (2001); Aronov et al. (1999)).

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially, free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The term “antisense DNA” is the non-coding strand complementary to the coding strand in double-stranded DNA.

The term “genomic DNA” as used herein means all DNA from a subject including coding and non-coding DNA, and DNA contained in introns and exons.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid).

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The term “polymorphism” as used herein means the occurrence in the same population of multiple discrete allelic states of which at least two have high frequency (conventionally of 1% or more).

The term “single nucleotide polymorphism” or “small nucleotide polymorphism” as used in this application means a variation in DNA sequence at a single nucleotide.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, or GCG (Genetics Computer Group, Program Manual for the GCG Package, Version 7, Madison, Wis.).

The terms “substantially homologous” or “substantially similar” when at least about 80%, and most preferably at least about 90 or 95%, 96%, 97%, 98%, or 99% of the nucleotides match over the defined length of the DNA sequences, as determined by sequence comparison algorithms, such as BLAST, FASTA, and DNA Strider. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Biomarkers for Tangle-Predominant Dementia

TPD is a poorly-understood and under-recognized tauopathy in need of a definitive neuropathological designation. Given the overlapping features with moderate AD and aging, and the absence of reliable markers, recognizing TPD continues to pose a challenge. However, because TPD is Aβ independent, it is crucial to diagnose TPD correctly, and more specifically differentiate the disease from classical Alzheimer's disease so that the correct therapeutic interventions can be sought. As stated above, the key biomarkers for MCI due to AD are positive or are predicted to be positive (i.e., radiographically identified hippocampal atrophy, memory impairment/medial temporal lobe symptoms, low CSF Aβ and elevated CSF phospho-tau) in TPD. Thus, improved methodology to identify and treat TPD would be highly clinically useful.

The results set forth herein for the first time provide biochemical and genetic biomarkers for TPD. These biomarkers will allow the correct diagnosis of TPD in a subject exhibiting MCI. Furthermore, these biochemical and genetic biomarkers will allow a complete understanding of the mechanism of TPD, AD, other taupathies such as Parkinson's disease, as well as neurodegeneration in general.

Moreover, the data reported herein support the argument that TPD is more prevalent than recognized (Example 2), which is consistent with previous reports (Bancher and Jellinger (1994); Ikeda et al. (1997); Ulrich et al. (1992)). Should estimates of 3-5% of dementia patients prove accurate, TPD may be among the more common neurodegenerative disorders, affecting between 1.1 and 1.8 million individuals globally.

Post-mortem examination of the brains of TPD patients revealed a pattern reminiscent of early to moderate-stage AD. There was gross medial temporal lobe atrophy compared to controls. Unlike most late-stage AD patients, frontal, parietal and occipital cortices are preserved in TPD. Microscopically, TPD brains exhibited severe medial temporal lobe tauopathy with frequent NFT, and the NFT were immunopositive for 3R and 4R tau as well as for various phospho-tau specific epitopes, which is the same profile as seen in AD and certain rare tauopathies (Ikeda et al. (1999); Iseki et al. (1997); Noda et al. (2006)). Moreover, it was confirmed that extracellular NFT have disproportionate immunolabeling for 3R tau in TPD, as previously reported (Iseki et al. (2006)). Electron microscopy of the cornu ammonis 1 (CA1) sector of the hippocampal formation showed paired-helical filaments (PRFs) in TPD, as is observed in AD (Kidd (1963)). However, no insoluble amyloid deposits were found in TPD patients (Example 2). Biochemical analysis of tau in both AD and TPD showed little to no differences (Example 3). Taken together, the data show that the NFT in TPD are regionally, histologically, biochemically and ultrastructurally similar to those in early to moderate-stage AD. However, differences between AD and TPD were seen in the biochemical characterization of Aβ and APP. Aβ is derived from the amyloid precursor protein (APP) (Thinakaren and Koo (2008)). Proteolysis of APP by a-secretase or β-secretase produces secreted N-terminal fragments termed sAPPα and sAPPβ respectively as well as C-terminal fragments (CTFs). Cleavage by γ-secretase of the f3-CTF yields Aβ peptides of predominantly 40 or 42 amino acids.

There was no significant Aβ deposition in TPD and lower levels of soluble Aβ in TPD brains when compared to AD. The control brains have variable levels of soluble Aβ that overlap with those observed in AD and TPD (Example 4).

Low Aβ may arise from decreased production, decreased fibrillization or increased clearance. Decreased levels of full-length APP in AD as well were observed, consistent with previous reports (Davidisson et al. (2001); Wu et al. (2011)). TPD is unlike AD in that BA9 is preserved, leading to the conclusions that low APP levels in TPD reflect differences in underlying APP metabolism rather than neuronal loss and gliosis. Significantly lower sAPPβ levels, and significantly higher sAPPα levels, were found in TPD patients, as compared to controls (Example 4).

Finally, there is no difference in the levels of APP mRNA among TPD, AD and control in BA9, suggesting that non-amyloidogenic processing contributes to decreased production of Aβ in TPD (Example 4).

Apolipoprotein E (ApoE) alleles correlate with AD risk and amyloid plaque load (Corder et al. (2006); Saunders et al. (1993)). The ApoE allele frequency was looked at and there was a significant decrease in ε4 in TPD as compared to age-matched AD and an increase in ε2 and ε3, (Example 4), which is consistent with previous studies (Ikeda et al. (1999); Jellinger and Bancher (1998)). These data support the well-established finding that ApoE ε4 is associated with Aβ deposition and ε2 is protective (Corder et al. (1993)).

Thus, the discordance in the spatial and temporal development of amyloid plaques and medial temporal lobe NFT suggest that these two pathologies may have distinct pathogenic mediators (Price and Morris (1999)). The findings presented here strongly suggest that toxicity from non-fibrillar soluble Aβ does not contribute to TOD. This conclusion is consistent with the absence of significant amyloid in TOD brain parenchyma when assessed biochemically and immunohistochemically as well as the absence of an association with the ApoE A ε4 allele, which is strongly associated with Aβ deposition (Corder et al. (1993); Saunders et al. (1993)). The observation that TOD patients have an increase in sAPPα provides additional evidence that the amyloidogenic pathway is not dominant in these patients. Increased sAPPα may have a partially protective influence, as sAPPα attenuates excitotoxicity and Aβ-induced tau phosphorylation (Mattson et al. (1993); Stein et al. (2004)), perhaps serving to delay the onset and severity of TOD relative to AD. In this context, this data are consistent with a role for amyloid in accelerating and amplifying an age-related tauopathy, perhaps by influencing trans-synaptic tau spreading.

Next it was asked whether TPD is associated with changes in the tau gene (MAPT). MAPT is within an approximately 900 kb ancestral genomic inversion that defines two haplotypes, H1 and H2 (Stefansson et al. (2005)). These haplotypes are in complete linkage disequilibrium and do not recombine. Sporadic tauopathies such as progressive supranuclear palsy and corticobasal degeneration as well as Parkinson disease are associated with the H1 haplotype (Baker et al. (1999); Bekris et al. (2010); DiMaria et al (2000)). There are conflicting reports concerning an association of MAPT with AD (Abraham et al. (2009); Mukherjee et al. (2007); Myers et al. (2005)). How H1 confers risk for tauopathy is unclear, but increased expression of 4R tau mRNA isoforms has been implicated (Myers et al (2007)), albeit controversially (Hayesmoore et al. (2009)). Other factors may play a role. For example, elements in the tau 3′ UTR regulate mRNA stability and localization leading to speculation that polymorphisms in this region underlie disease risk (Aronov et al. (1999); Aronov et al. (1999); Vandrovcova et al. (2010)). The approximate 2 Mb H1 haplotype is found on chromosome 17 between base pairs 43,000,000 and 45,000,000, and is obtainable in the genome browser at chr17:43,000,000-45,000,000 (UCSC Genome Browser on Human February 2009 (GRCh37/hg19) Assembly). The H1 haplotype of the MAPT 3′ UTR is found at chromosome 17 between base pairs 44,101295 and 44,105,727 and is set forth in SEQ ID NO: 6.

The results shown herein confirm that H1 is associated with TPD (Example 5). Additionally, two polymorphisms within the region of MAPT encoding the 3′ UTR were identified that are significantly associated with TPD, rs5820605 and rs35134656 (Example 6).

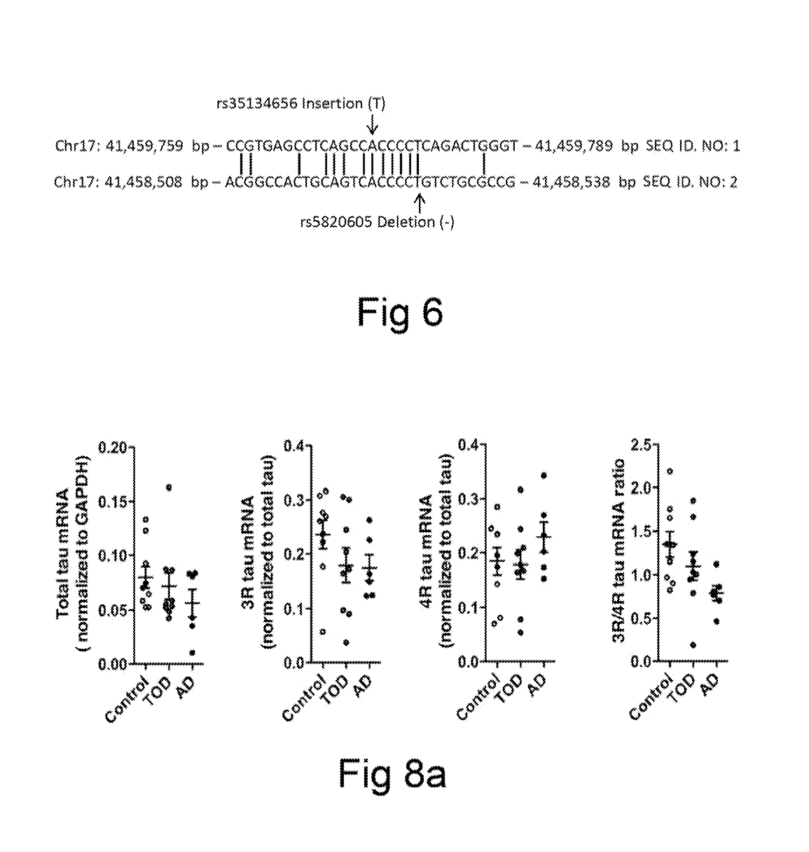

3′ UTRs are critical cis-acting regulatory elements that are capable of regulating gene expression on the post-transcriptional level by influencing mRNA stability and localization, among other functions (Aronov et al. (2001); Aronov et al. (1999)). These two distinct polymorphisms, one predicted to confer risk, rs35134656, found in TPD patients three times more than in controls, and the other protective, rs5820605, are contained within a 10 bp motif with high sequence identity and high conservation, suggesting that the motif is functional. The finding that a single nucleotide deletion in this motif at rs5820605 suppresses baseline tau 3′ UTR activity in vitro compared to control H1 and H2 3′ UTRs suggests that alterations in tau expression underlie tauopathy risk. The increased tau 3′ UTR activity that was observed following Aβ is not impacted by rs5820605, suggesting that Aβ responsive elements lie outside this variant (Example 7). The sequences of these polymorphisms, as well as the position of the mutations and conserved 10 bp motif are shown in FIG. 6. Together, these finding suggest that dysregulation of complex expression programs may underlie TOD and AD.

The ultimate cause of TPD is unclear at this time. The increased levels of sAPPα in TPD is a surprising finding. Previous research suggests that sAPPα attenuates excitotoxicity and Aβ-induced tau phosphorylation, perhaps serving to delay the onset and severity of TPD relative to AD (Mattson et al. (1993)). In this context, the data set forth herein are consistent with a proposed role for Aγ in accelerating and amplifying an age-related tauopathy (Purcell et al. (2007)).

Previous work has also suggested a viral etiology for TPD (Nelson et al. (2010)), as well as other potential causes of neurofibrillary degeneration, such as mild traumatic injury (Gavett et al. (2010)). The regional overlap of NFT between TPD and aging has prompted some investigators to suggest TPD represents “pathological” aging (Jellinger and Bancher (1998)).

Nevertheless, currently, there is no way to clinically differentiate TPD from classical plaque and tangle AD, yet the distinction is critical for implementing Aβ-targeted therapies. Given the low Aβ levels in TPD, it is unlikely that Aβ-targeted agents will be useful and they may subject TOD patients to unnecessary risk. Given the predominantly amnestic symptoms, TOD patients may be clinically classified having AD (Albert et al. (2011)).

The data set forth herein for the first time shows biomarkers specific for TPD that can be used to diagnose TPD in a subject clinically classified as MCI. Specifically, TPD can be characterized by:
Decreased levels of Aβ;
Increased levels of sAPPα;
Decreased levels of sAPPβ;
Increased association of the ApoE allele ε2;
Increased association of the ApoE allele ε3;
Decreased association of the ApoE allele ε4;
Increased association with the H1 haplotype;
Decreased association of the polymorphism rs5820605 in the 3′UTR of MAPT; and
Increased association of the polymorphism rs35134656 in the 3′UTR of MAPT.
   Thus, any or all of these characteristics can be used as biomarkers for the screening, the diagnosing, predicting, and/or identifying of TPD, and in particular, distinguishing TPD from AD.

In a preferred embodiment of the methods of screening and diagnosis, a subject with cognitive impairment could be tested for all of the amyloid characteristics, i.e., Aβ peptide, sAPPα peptide, sAPPβ peptide, ApoE allele ε2, ApoE allele ε3 and ApoE allele ε4. A finding that the subject has decreased levels or low quantities of Aβ peptide, and sAPPβ peptide, along with increased levels or high quantities of sAPPα, peptide and the ε2 or ε3 allele, would indicate the subject has TPD.

In another preferred embodiment, a subject with cognitive impairment could be tested for all of the tau characteristics, i.e., the H1 haplotype and the polymorphisms. A finding of the H1 haplotype along with the polymorphism designated rs35234656 would indicate the subject has TPD.

### Use of Levels of Aβ as a Screening and Diagnosis Method for TPD

As stated above, and shown in Example 4, TPD is associated with lower levels of Aβ protein or peptide. Thus, one embodiment of the present invention is the screening, diagnosis, prediction or identification of tangle-predominant dementia in a subject, by detection and/or measurement of decreased levels or quantity of Aβ in a sample from a subject with cognitive impairment.

A sample of biological tissue or bodily fluid from a subject with cognitive impairment ranging from mild to severe, or with pre-dementia in the prodromal phase, is obtained.

The protein sample can be obtained from any biological tissue. Preferred biological tissues include, but are not limited to, brain, epidermal, whole blood, and plasma.

The protein sample can be obtained from any bodily fluid. Preferred bodily fluids include, but are not limited to, cerebrospinal fluid, plasma, saliva, sweat, and urine.

Protein is isolated and/or purified from the sample using any method known in the art including but not limited to the one described in Example 4. Other methods for protein isolation and purification include but are not limited to immunoaffinity chromatography.

Any method known in the art can be used, but preferred methods for detecting and measuring decreased levels of Aβ in a protein sample include quantitative Western blot, immunoblot, quantitative mass spectrometry, enzyme-linked immunosorbent assays (ELISAs), radioimmunoassays (RIA), immunoradiometric assays (IRMA), and immunoenzymatic assays (TEMA) and sandwich assays using monoclonal and polyclonal antibodies.

Antibodies are a preferred method of detecting and measuring Aβ in a sample. Such antibodies are available commercially (Covance) or can be made by conventional methods known in the art. Such antibodies can be monoclonal or polyclonal and fragments thereof, and immunologic binding equivalents thereof. The term “antibody” means both a homologous molecular entity as well as a mixture, such as a serum product made up of several homologous molecular entities.

In a preferred embodiment, such antibodies will immunoprecipitate Aβ peptide from a solution as well as react with Aβ peptide on a Western blot, immunoblot, ELISA, and other assays listed above. In another preferred embodiment, these antibodies will react and detect Aβ peptide in frozen tissue section, say from a brain biopsy.

Antibodies for use in these assays can be labeled covalently or non-covalently with an agent that provides a detectable signal. Any label and conjugation method known in the art can be used. Labels, include but are not limited to, enzymes, fluorescent agents, radiolabels, substrates, inhibitors, cofactors, magnetic particles, and chemiluminescent agents.

The levels or quantity of Aβ peptide found in a sample are compared to the levels or quantity of the peptide in healthy controls and/or patients known to have AD and a deviation in the level or quantity of peptide is looked for. This comparison can be done in many ways. The same assay can be performed simultaneously or consecutively, on a purified and/or isolated protein sample from a healthy control and/or an AD patient, and the results compared qualitatively, e.g., visually, i.e., does the protein sample from the healthy control and/or the AD patient, produce the same intensity of signal as the protein sample from the subject in the same assay, or the results can be compared quantitatively, e.g., a value of the signal for the protein sample from the subject is obtained and compared to a known reference value of the protein in a healthy control and/or the patient with AD.

A lower level or quantity of Aβ peptide in a sample from a subject as compared to the reference value of the level or quantity of Aβ peptide of healthy control and/or a patient known to have AD, would indicate or predict that the subject has TPD.

In a preferred embodiment, the level of Aβ42 is detected or measured in the protein sample by any of the methods set forth above, and compared to the levels or quantity of the reference value of the peptide in healthy controls and/or patients known to have AD. In a further preferred embodiment, the level of Aβ40 is detected or measured in the protein sample by any of the methods set forth above, and compared to the levels or quantity of the reference value of the peptide in healthy controls and/or patients known to have AD. In the most preferred embodiment, the levels of both peptides are detected or measured and a ratio of the level of Aβ42/Aβ40 is determined. In all these embodiments, if the level or quantity of Aβ42, the level or quantity of Aβ40, and/or the ratio of Aβ42/Aβ40, is lower than the levels or ratio in healthy controls and/or patients with AD, the subject would be predicted, identified or diagnosed with TPD.

Use of Levels of sAPP as a Screening and Diagnosis Method for TPD

As stated above, and shown in Example 4, TPD is associated with higher levels of sAPPα and lower levels of sAPPβ. Thus, one embodiment of the present invention is the screening, diagnosis, prediction or identification of tangle-predominant dementia in a subject, by detection of increased levels or quantities of sAPPα in a sample from a subject with cognitive impairment. Another embodiment of the present invention is the screening, diagnosis, prediction or identification of tangle-predominant dementia in a subject, by the detection of decreased levels or quantities of sAPPβ in a sample from a subject with cognitive impairment.

A sample of biological tissue or bodily fluid from a subject with cognitive impairment ranging from mild to severe, or with pre-dementia in the prodromal phase, is obtained.

The protein sample can be obtained from any biological tissue. Preferred biological tissues include, but are not limited to, brain, epidermal, whole blood, and plasma.

The protein sample can be obtained from any bodily fluid. Preferred bodily fluids include, but are not limited to, cerebrospinal fluid, plama, saliva, sweat, and urine.

Protein is purified and/or isolated from the sample using any method known in the art including but not limited to the one described in Example 4. Other methods for protein purification and isolation include but are not limited to immunoaffinity chromatography.

Any method known in the art can be used, but preferred methods for detecting increased levels or quantities of sAPPα and/or decreased levels or quantities of sAPPβ in a protein sample include quantitative Western blot, immunoblot, quantitative mass spectrometry, enzyme-linked immunosorbent assays (ELISAs), radioimmunoassays (RIA), immunoradiometric assays (IRMA), and immunoenzymatic assays (IRMA) and sandwich assays using monoclonal and polyclonal antibodies.

Antibodies are a preferred method of detecting sAPPα and/or sAPPβ in a sample. Such antibodies are available commercially (American Research Products; Covance) or can be made by conventional methods known in the art. Such antibodies can be monoclonal or polyclonal and fragments thereof, and immunologic binding equivalents thereof. The term “antibody” means both a homologous molecular entity as well as a mixture, such as a serum product made up of several homologous molecular entities.

In a preferred embodiment, such antibodies will immunoprecipitate sAPPα and/or sAPPβ peptide from a solution as well as react with sAPPα and/or sAPPβ peptide on a Western blot, or immunoblot, ELISA, and other assays listed above. In another preferred embodiment, these antibodies will react and detect sAPPα and/or sAPPβ peptide in frozen tissue section, say from a brain biopsy.

Antibodies for use in these assays can be labeled covalently or non-covalently with an agent that provides a detectable signal. Any label and conjugation method known in the art can be used. Labels, include but are not limited to, enzymes, fluorescent agents, radiolabels, substrates, inhibitors, cofactors, magnetic particles, and chemiluminescent agents.

The levels or quantities of sAPPα and/or sAPPβ peptide found in a sample are compared to the levels or quantities of these peptides in healthy controls and a deviation in the level or quantity of peptides is looked for. This comparison can be done in many ways. The same assay can be performed simultaneously or consecutively, on a purified and/or isolated protein sample from a healthy control and the results compared qualitatively, e.g., visually, i.e., does the protein sample from the healthy control produce the same intensity of signal as the protein sample from the subject in the same assay, or the results can be compared quantitatively, e.g., a value of the signal for the protein sample from the subject is obtained and compared to a known reference value of the protein in a healthy control.

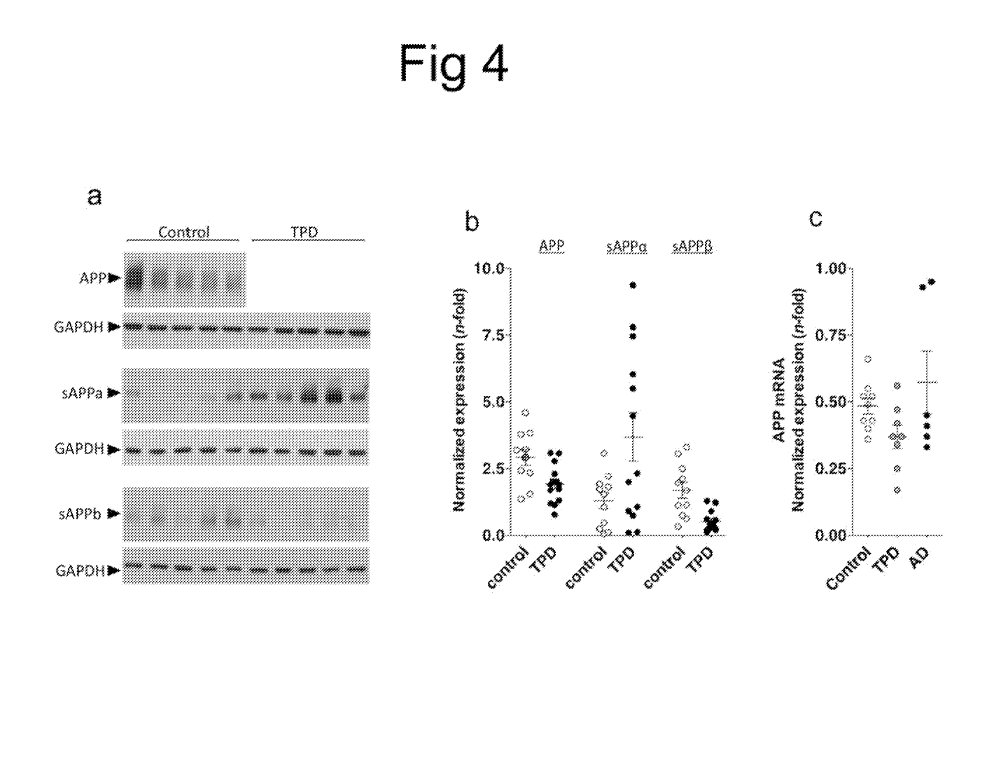

Alternatively, as shown in Example 4 and FIG. 4a, in a protein sample of a patient with TPD, there was clearly a signal when hybridized with sAPPα antibody, and clearly not a signal when hybridized with sAPPβ antibody.

A higher level or quantity of sAPPα and/or the lower level or quantity of sAPPβ peptide in a sample from a subject as compared to the reference value of the level or quantity of the peptides in a healthy control would indicate the subject has TPD. In a preferred embodiment, the sample is tested for levels or quantities of both sAPPα and/or sAPPβ peptide.

### Use of Apo E Alleles as a Screening and Diagnosis Method for TPD

As stated above and shown in Example 4, TPD is associated with the presence or absence of Apo E alleles. This association can be used to screen for, diagnose or identify TPD.

In order to detect the Apo E alleles associated with tangle-predominant dementia, a biological sample from a subject with cognitive impairment ranging from mild to severe, or with pre-dementia in the prodromal phase is obtained and prepared and analyzed for the presence of the Apo E alleles ε2, ε3, and/or ε4. This can be achieved in numerous ways, by a diagnostic laboratory, and/or a health care provider. Specifically the presence of ε2 and ε3, and/or the absence of ε4 would indicate a diagnosis of TPD.

Any method known in the art can be used to detect the presence or absence of the Apo E alleles. Preferred methods that can be utilized in this analysis are sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis.

DNA Encoding the Apo E Alleles

There are three Apo E alleles associated with TPD-82, 0, and 84.

The sequences of at least the ε2 and ε4 alleles are known. The SNP rs7412 comprises the sequence of the ε2 allele (SEQ ID NO: 7), and SNP rs429358 comprises the sequence of the ε4 allele (SEQ ID NO: 8). Using these two sequences alone is enough to determine which Apo E allele a subject possesses. In other words, if the subject is not positive for the ε2 or ε4 allele, they must possess the ε3 allele.

Such DNA sequences, no matter how obtained, are useful in the methods set forth herein for diagnosing TPD. In the simplest embodiment of the present invention DNA isolated and prepared from a sample of biological tissue and/or bodily fluid from a subject with cognitive impairment ranging from mild to severe, or with pre-dementia in the prodromal phase is compared to the known sequences of the Apo E alleles, specifically to the polymorphisms designated rs7412 and rs429358, to screen for, predict, or confirm a diagnosis of TPD.

The isolated DNA can also be used as the basis for probes and primers for used in additional diagnostic procedures for TPD.

### Use of H1 Haplotype as a Screening and Diagnosis Method for TPD

As stated above and shown in Example 5, TPD is closely associated with the H1 haplotype of the MAPT locus. Thus, a further embodiment of the present invention is the use of this association to screen for, predict, diagnose or identify TPD.

In order to detect the H1 haplotype associated with tangle-predominant dementia, a biological sample from a subject with cognitive impairment ranging from mild to severe, or with pre-dementia in the prodromal phase is obtained and prepared and analyzed for the presence of the H1 haplotype, and/or the presence of the H1 subhaplotypes H1b, H1C, H1d, and H1e, as set forth in Table 9. This can be achieved in numerous ways, by a diagnostic laboratory, and/or a health care provider.

Any method known in the art can be used to detect the presence or absence of the H1 haplotype. Preferred methods that can be utilized in this analysis are sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis.

DNA Encoding the H1 Haplotype and the H1 Subhaplotypes

One embodiment of the present invention is the use of the isolated DNA encoding the H1 haplotype of the MAPT gene, found on chromosome 17 between base pairs 43,000,000 and 45,000,000, and obtainable in the genome browser at chr17:43,000,000-45,000,000 (UCSC Genome Browser on Human February 2009 (GRCh37/hg19) Assembly), as a diagnostic for TPD.

Further embodiments of the present invention are methods using the isolated DNA of the H1 subhaplotype alleles set forth in Table 9.

Further embodiments of the present invention are methods of using the isolated DNA of the H1 haplotype of the 3′ UTR of the MAPT gene comprising the nucleotide sequence of SEQ ID NO: 6.

The present invention also includes the use of the antisense DNA of the H1 haplotype, as well as the DNA sequences listed in Table 9 and SEQ ID NO: 6.

The present invention also includes recombinant constructs comprising the DNA comprising the nucleotide sequence of H1 haplotype of the MAPT gene, SEQ ID NO: 6, or the sequences in Table 9 for the H1 subhaplotypes, or the antisense DNA comprising the nucleotide sequence of H1 haplotype of the MAPT gene, SEQ ID NO: 6, or the sequences in Table 9 for the H1 subhaplotypes, and a vector, that can be expressed in a transformed host cell. The present invention also includes the host cells transformed with the recombinant construct comprising DNA comprising the nucleotide sequence of H1 haplotype of the MAPT gene, SEQ ID NO: 6, or the sequences in Table 9 for the H1 subhaplotypes, or the antisense DNA comprising the nucleotide sequence of H1 haplotype of the MAPT gene, SEQ ID NO: 6, or the sequences in Table 9 for the H1 subhaplotypes, and a vector.

Such DNA sequences, no matter how obtained, are useful in the methods set forth herein for diagnosing TPD. In the simplest embodiment of the present invention, DNA isolated and prepared from a sample of biological tissue and/or bodily fluid from a subject with cognitive impairment ranging from mild to severe, or with pre-dementia in the prodromal phase is compared to the DNA sequence of the H1 haplotype of the MAPT gene, SEQ ID NO: 6 and/or any of the sequences in Table 9 that correspond to the H1 subhaplotype to confirm a diagnosis of TPD.

The isolated DNA can also be used as the basis for probes and primers for used in additional diagnostic procedures for TPD.

### Use of Polymorphisms in the MAPT 3′UTR as a Screening and Diagnosis Method for TPD

As shown by the data herein, there are two polymorphisms within the 3′UTR region of MAPT that are closely associated with TPD. These polymorphisms, designated rs5820605 and rs35134656, can be used as a diagnosis for TPD. Additionally, there is a conserved 11 bp motif (CAGNCACCCCT) (SEQ ID NO: 3) contained in these two polymorphisms.

In order to detect one of the polymorphisms associated with tangle-predominant dementia, a biological sample from a subject with cognitive impairment ranging from mild to severe, or with pre-dementia in the prodromal phase is obtained and prepared and analyzed for the presence of polymorphism rs35134656, and/or the absence of rs5820605. This can be achieved in numerous ways, by a diagnostic laboratory, and/or a health care provider.

Any method known in the art can be used to detect the presence or absence of the polymorphisms. Preferred methods that can be utilized in this analysis are sequencing, hybridization with probes including Southern blot analysis and dot blot analysis, polymerase chain reaction (PCR), PCR with melting curve analysis, PCR with mass spectrometry, fluorescent in situ hybridization, DNA microarrays, single-strand conformation analysis, and restriction length polymorphism analysis.

DNA Encoding MAPT 3′UTR Polymorphisms

One embodiment of the present invention is the use of the isolated DNA encoding the MAPT 3′UTR polymorphism, rs35134656, comprising the nucleotide sequence of SEQ ID NO. 1.

A further embodiment of the present invention is the use of the isolated DNA sequence encoding the MAPT 3′ UTR polymorphism, rs5820605, comprising the nucleotide sequence SEQ ID NO: 2.

Another embodiment of the present invention is the use of the isolated conserved 11 base pair DNA from the MAPT 3′UTR, comprising the nucleotide sequence of SEQ ID NO: 3.

The present invention also includes the use of the antisense DNA of SEQ ID NOs: 1, 2, and 3.

The present invention also includes recombinant constructs comprising the DNA having the nucleotide sequence of SEQ ID NOs: 1, 2, and/or 3 or the antisense DNA of SEQ ID NOs: 1, 2, and/or 3, and a vector, that can be expressed in a transformed host cell. The present invention also includes the host cells transformed with the recombinant construct comprising DNA having the nucleotide sequence of SEQ ID NOs: 1, 2, and/or 3 or the antisense DNA of SEQ ID NOs: 1, 2, and/or 3 and a vector.

Such DNA sequences, no matter how obtained, are useful in the methods set forth herein for diagnosing TPD. In the simplest embodiment of the present invention DNA isolated and prepared from a sample of biological tissue or bodily fluid from a subject with ranging from mild to severe, or with pre-dementia in the prodromal phase is compared to the DNA SEQ ID NOs: 1 and/or 2 to confirm a diagnosis of TPD.

The isolated DNA can also be used as the basis for probes and primers for used in additional diagnostic procedures for TPD.

### Probes and Primers

Further embodiments of the present invention include probes comprising some or all of the DNA comprising the nucleotide sequence of SEQ ID NOs: 7 and 8, and probes comprising some or all of the DNA with the antisense nucleotide sequence of SEQ ID NOs: 7 and 8. These probes can be used to detect Apo E alleles associated with TPD in a sample of DNA from a subject with cognitive impairment, and confirm a diagnosis of TPD in a subject with cognitive impairment.

Further embodiments of the present invention include probes comprising some or all of the DNA comprising the nucleotide sequence of the H1 haplotype of the MAPT locus, SEQ ID NO: 6 and the sequences in Table 9, and probes comprising some or all of the DNA comprising the antisense nucleotide sequence of H1 haplotype of the MAPT locus, SEQ ID NO: 6 and the sequences in Table 9. These probes can be used to detect H1 haplotype and subhaplotypes associated with TPD in a sample of DNA from a subject with cognitive impairment, and confirm a diagnosis of TPD in a subject with cognitive impairment.

Further embodiments of the present invention include probes comprising some or all of the DNA comprising the nucleotide sequence of SEQ ID NOs: 1, 2, and 3, and probes comprising some or all of the DNA comprising the antisense nucleotide sequence of SEQ ID NOs: 1, 2, and 3. These probes can be used to detect the polymorphisms and/or conserved 11 bp motif associated with TPD in a sample of DNA from a subject with cognitive impairment, and confirm a diagnosis of TPD in a subject with cognitive impairment.

Probes contemplated for use in the screening and diagnostic assays of the present invention can be made by any method known in the art, including the procedures outlined below.

In standard nucleic acid hybridization assays, probe must be is labeled in some way, and must be single stranded. Oligonucleotide probes are short (typically 15-50 nucleotides) single-stranded pieces of DNA made by chemical synthesis: mononucleotides are added, one at a time, to a starting mononucleotide, conventionally the 3′ end nucleotide, which is bound to a solid support. Generally, oligonucleotide probes are designed with a specific sequence chosen in response to prior information about the target DNA. Oligonucleotide probes are often labeled by incorporating a ³²P atom or other labeled group at the 5′ end.

Conventional DNA probes are isolated by cell-based DNA cloning or by PCR. In the former case, the starting DNA may range in size from 0.1 kb to hundreds of kilobases in length and is usually (but not always) originally double-stranded. PCR-derived DNA probes have often been less than 10 kb long and are usually, but not always, originally double-stranded. DNA probes are usually labeled by incorporating labeled dNTPs during an in vitro DNA synthesis reaction by many different methods including nick-translation, random primed labeling, PCR labeling or end-labeling.

Labels can be radioisotopes such as ³²P, ³³P, ³⁵S and ³H, which can be detected specifically in solution or, more commonly, within a solid specimen, such as autoradiography. ³²P has been used widely in Southern blot hybridization, and dot-blot hybridization.

Nonisotopic labeling systems which use nonradioactive probes can also be used in the current invention. Two types of non-radioactive labeling include direct nonisotopic labeling, such as one involving the incorporation of modified nucleotides containing a fluorophore. The other type is indirect nonisotopic labeling, usually featuring the chemical coupling of a modified reporter molecule to a nucleotide precursor. After incorporation into DNA, the reporter groups can be specifically bound by an affinity molecule, a protein or other ligand which has a very high affinity for the reporter group. Conjugated to the latter is a marker molecule or group which can be detected in a suitable assay. This type of labeling would include biotin-streptavidin and digoxigenin.

Primers for use in the various assays of the present invention are also an embodiment of the present invention. A forward primer for amplifying the full-length MAPT 3′UTR having the nucleotide sequence of SEQ ID NO: 4 is one embodiment of the invention, and a reverse primer for amplifying the full-length MAPT 3′UTR having the nucleotide sequence of SEQ ID NO: 5 is yet another embodiment of the present invention.

Additionally other primers useful for the methods of screening and diagnosis of the present invention are also contemplated by the invention and can be prepared by method known in the art as outlined below, using the sequences of the MAPT 3′UTR, and H1 haplotype of the MAPT gene, as well as the sequences of the Apo E alleles, e.g., polymorphisms rs429358 and rs7412, and the polymorphisms rs5820605 and rs35134656.

The specificity of amplification depends on the extent to which the primers can recognize and bind to sequences other than the intended target DNA sequences. For complex DNA sources, such as total genomic DNA from a mammalian cell, it is often sufficient to design two primers about 20 nucleotides long. This is because the chance of an accidental perfect match elsewhere in the genome for either one of the primers is extremely low, and for both sequences to occur by chance in close proximity in the specified direction is normally exceedingly low. Although conditions are usually chosen to ensure that only strongly matched primer-target duplexes are stable, spurious amplification products can nevertheless be observed. This can happen if one or both chosen primer sequences contain part of a repetitive DNA sequence, and primers are usually designed to avoid matching to known repetitive DNA sequences, including large runs of a single nucleotide

After the primers are added to denatured template DNA, they bind specifically to complementary DNA sequences at the target site. In the presence of a suitably heat-stable DNA polymerase and DNA precursors (the four deoxynucleoside triphosphates, dATP, dCTP, dGTP and dTTP), they initiate the synthesis of new DNA strands which are complementary to the individual DNA strands of the target DNA segment, and which will overlap each other.

### Screening and Diagnostic Assays

Several methods can be used to screen for, diagnose, predict, and identify TPD in a subject with cognitive impairment utilizing the surprising discoveries of the association of certain Apo E alleles, and the H1 haplotype and the two polymorphisms in the MAPT 3′UTR with TPD, as well as a conserved 11 base pair motif in these patients.

The most direct method for screening for and diagnosing TPD in a patient with cognitive impairment ranging from mild to severe, or with pre-dementia in the prodromal phase, is to obtain a sample of biological tissue or bodily fluid from the patient and extracting, isolating and/or purifying the nucleic acid (e.g., genomic DNA, cDNA, RNA) from the tissue or fluid.

The nucleic acid can be obtained from any biological tissue. Preferred biological tissues include, but are not limited to, brain, epidermal, whole blood, and plasma.

The nucleic acid can be obtained from any bodily fluid. Preferred bodily fluids include, but are not limited to, cerebrospinal fluid, plasma, saliva, sweat, and urine.

The nucleic acid is extracted, isolated and purified from the cells of the tissue or fluid by methods known in the art. The nucleic acid, e.g., DNA is then sequenced.

In one embodiment, the nucleic acid is sequenced at the Apo E locus, and the sequenced nucleic acid is then inspected at the Apo E locus for the Apo E alleles. Specifically, the DNA from the patient is compared to the DNA of one or all of the nucleotides comprising the sequences of one of, or both SEQ ID NOs: 7 and 8. The comparison can be made to one sequence, or most preferably both sequences. The presence of the ε2 allele set forth in SEQ ID NO: 7, and/or the absence of the ε4 allele set forth in SEQ ID NO: 8 would indicate the patient has TPD.

In another embodiment, the nucleic acid is sequenced at the MAPT locus and the sequenced nucleic acid is inspected at the MAPT locus for either the H1 haplotype and/or any of the H1 subhaplotypes. Specifically, the isolated, purified and sequenced DNA from the patient is compared to the DNA with the nucleotide sequences of one or all of the H1 haplotype found on chromosome 17 between base pairs 43,000,000 and 45,000,000, and obtainable in the genome browser at chr17:43,000,000-45,000,000 (UCSC Genome Browser on Human February 2009 (GRCh37/hg19) Assembly), SEQ ID NOs: 6, and the sequences for the H1 subhaplotypes in Table 9. The comparison can be made to one sequence, two sequences, three sequences, four sequences, five sequences, or preferably all six. The presence of any of these DNA sequences in the DNA from the biological tissue or fluid of the subject would indicate the subject has TPD.

Another direct method for screening for or diagnosing TPD in a patient is to inspect the nucleic acid sequenced at the MAPT 3′UTR locus for the either of the polymorphisms, and/or the conserved 11 bp motif. Specifically, the isolated, purified and sequenced DNA from the patient is compared to the DNA with the nucleotide sequences of SEQ ID NOs: 1 and/or 2. The comparison can be made to one sequence or both sequences. The presence of the rs35134565 polymorphism set forth in SEQ ID NO: 1, and/or the absence of the rs5820605 polymorphism set forth in SEQ ID NO: 2 would indicate the subject has TPD.

The DNA from the subject can be sequenced by direct DNA sequencing either manual or automated by methods known in the art such as Sanger sequencing, dideoxy sequencing, and automated fluorescent sequencing.

Screening and diagnostic method of the current invention may involve the amplification of the MAPT locus, the 3′UTR of the MAPT locus, or the amplification of the Apo E locus. A preferred method for target amplification of nucleic acid sequences is using polymerases, in particular polymerase chain reaction (PCR). PCR or other polymerase-driven amplification methods obtain millions of copies of the relevant nucleic acid sequences which then can be used as substrates for probes or sequenced or used in other assays. An example of PCR is found in Example 7 and primers with the nucleotide sequences SEQ ID NOs: 4 and 5 would be useful in embodiments of the present invention.

Amplification using polymerase chain reaction is particularly useful in the embodiments of the current invention. PCR is a rapid and versatile in vitro method for amplifying defined target DNA sequences present within a source of DNA. Usually, the method is designed to permit selective amplification of a specific target DNA sequence(s) within a heterogeneous collection of DNA sequences (e.g. total genomic DNA or a complex cDNA population). To permit such selective amplification, some prior DNA sequence information from the target sequences is required. This information is used to design two oligonucleotide primers (amplimers) which are specific for the target sequence and which are often about 15-25 nucleotides long.

Of particular usefulness in the current invention is the use of oligonucleotide primers to discriminate between target DNA sequences that differ by a single nucleotide in the region of interest called allele-specific PCR. These allele-specific primers will anneal only to the alleles of interest. In this case, the primers of the current invention made from the nucleotide sequence of the H1 haplotype found on chromosome 17 between base pairs 43,000,000 and 45,000,000, and obtainable in the genome browser at chr17:43,000,000-45,000,000 (UCSC Genome Browser on Human February 2009 (GRCh37/hg19) Assembly), and /or nucleotide sequence set forth in SEQ ID NO: 6 can be used as an initial screen of the genomic DNA from the subject. Only if the DNA contains the H1 haplotype of the MAPT locus will the primers anneal and amplify the product. Additional primers that target the specific polymorphisms can be designed using the sequence information in SEQ ID NOs: 1 and 2, as well as allele specific primers designed to anneal only to DNA with the particular polymorphism. This technique can also be used to determine which of the Apo E alleles are present in the Apo E gene of the subject.

Mutation detection using the 5′→3′ exonuclease activity of Taq DNA polymerase (TaqMan™ assay) can also be used as a screening and diagnostic method of the current invention. Such an assay involves hybridization of three primers, the third primer being intended to bind just downstream of one of the conventional primers which should be allele-specific. The additional primer carries a blocking group at the 3′ terminal nucleotide so that it cannot prime new DNA synthesis and at its 5′ end carries a labeled group. In modern versions of the assay, the label is a fluorogenic group and the third primer also carries a quencher group. If the upstream primer which is bound to the same strand is able to prime successfully, Taq DNA polymerase will extend a new DNA strand until it encounters the third primer in which case its 5′→3′ exonuclease will degrade the primer causing release of separate nucleotides containing the dye and the quencher, and an observable increase in fluorescence.

PCR with melting curve analysis can also be used with the disclosed biomarkers to screen for, identify and diagnose TPD. PCR with melting curve analysis is an extension of PCR where the fluorescence is monitored over time as the temperature changes. Duplexes melt as the temperature increases and the hybridization of both PCR products and probes can be monitored. The temperature-dependent dissociation between two DNA-strands can be measured using a DNA-intercalating fluorophore such as SYBR green, EvaGreen or fluorophore-labelled DNA probes. In the case of SYBR green (which fluoresces 1000-fold more intensely while intercalated in the minor groove of two strands of DNA), the dissociation of the DNA during heating is measurable by the large reduction in fluorescence that results. Alternatively, juxtapositioned probes (one featuring a fluorophore and the other, a suitable quencher) can be used to determine the complementarity of the probe to the target sequence. This technique is sensitive enough to detect single-nucleotide polymorphisms (SNP) and can distinguish between various alleles by virtue of the dissociation patterns produced.

PCR with mass spectrometry uses mass spectrometry to detect the end product. Primer pairs are used and tagged with molecules of known masses, known as MassCodes. If DNA from any of the agent of primer panel is present, it will be amplified. Each amplified product will carry its specific Masscodes. The PCR product is then purified to remove unbound primers, dNTPs, enzyme and other impurities. Finally, the purified PCR products are subject of ultraviolet as the chemical bond with nucleic acid and primers are photolabile. As the Masscodes are liberated from PCR products they are detected with a mass spectrometer.

When a probe is to be used to detect the presence of the Apo E alleles, H1 haplotype, the H1 subhaplotype alleles, the polymorphisms, rs35234656 and rs5820605 and/or the 11 bp conserved locus, the biological sample that is to be analyzed must be treated to extract the nucleic acids. The nucleic acids to be targeted usually need to be at least partially single-stranded in order to form a hybrid with the probe sequence. It the nucleic acid is single stranded, no denaturation is required. However, if the nucleic acid to be probed is double stranded, denaturation must be performed by any method known in the art.

The nucleic acid to be analyzed and the probe are incubated under conditions which promote stable hybrid formation of the target sequence in the probe and the target sequence in the nucleic acid. The desired stringency of the hybridization will depend on factors such as the uniqueness of the probe in the part of the genome being targeted, and can be altered by washing procedure, temperature, probe length and other conditions known in the art, as set forth in Maniatis et al. (1982) and Sambrook et al. (1989).

Labeled probes are used to detect the hybrid, or alternatively, the probe is bound to a ligand which labeled either directly or indirectly. Suitable labels and methods for labeling are known in the art, and include biotin, fluorescence, chemiluminescence, enzymes, and radioactivity.

Assays using such probes include Southern blot analysis. In such an assay, a patient sample is obtained, the DNA processed, denatured, separated on an agarose gel, and transferred to a membrane for hybridization with a probe. Following procedures known in the art (e.g., Sambrook et al. (1989)), the blots are hybridized with a labeled probe and a positive band indicates the presence of the target sequence. Southern blot hybridization can also be used to screen for the polymorphisms. In this method, the target DNA is digested with one or more restriction endonucleases, size-fractionated by agarose gel electrophoresis, denatured and transferred to a nitrocellulose or nylon membrane for hybridization. Following electrophoresis, the test DNA fragments are denatured in strong alkali. As agarose gels are fragile, and the DNA in them can diffuse within the gel, it is usual to transfer the denatured DNA fragments by blotting on to a durable nitrocellulose or nylon membrane, to which single-stranded DNA binds readily. The individual DNA fragments become immobilized on the membrane at positions which are a faithful record of the size separation achieved by agarose gel electrophoresis. Subsequently, the immobilized single-stranded target DNA sequences are allowed to associate with labeled single-stranded probe DNA. The probe will bind only to related DNA sequences in the target DNA, and their position on the membrane can be related back to the original gel in order to estimate their size.

Dot-blot hybridization can also be used to screen for the Apo E alleles, H1 haplotype and/or polymorphisms. Nucleic acid including genomic DNA, cDNA and RNA is obtained from the subject with suspected TPD, denatured and spotted onto a nitrocellulose or nylon membrane and lowed to dry. The membrane is exposed to a solution of labeled single stranded probe sequences and after allowing sufficient time for probe-target heteroduplexes to form, the probe solution is removed and the membrane washed, dried and exposed to an autoradiographic film. A positive spot is an indication of the target sequence in the DNA of the subject and a no spot an indication of the lack of the target sequence in the DNA of the subject.

A particularly useful application of dot blotting is the use of allele-specific oligonucleotide (ASO) probes. This method distinguishes between alleles that differ by even a single nucleotide substitution. ASO probes are using between 15-20 nucleotides long and are employed under hybridization conditions at which the DNA duplex between the probe and the target are stable only if there is a perfect base complementarity between them.

A further embodiment is the use of ASO reverse dot blotting, wherein an oligonucleotide probe is fixed on a filter or membrane and the target DNA is labeled and provided in a solution. Positive binding of labeled target DNA to a specific oligonucleotide on the membrane is taken to mean that the target DNA has the specific sequence.

DNA microarrays can also be used to screen for the H1 haplotype, Apo E alleles and polymorphisms. The surfaces involved are glass rather than porous membranes and similar to reverse dot-blotting, the DNA microarray technologies employ a reverse nucleic acid hybridization approach: the probes consist of unlabeled DNA fixed to a solid support (the arrays of DNA or oligonucleotides) and the target is labeled and in solution.

DNA microarray technology also permits an alternative approach to DNA sequencing by permitting by hybridization of the target DNA to a series of oligonucleotides of known sequence, usually about 7-8 nucleotides long. If the hybridization conditions are specific, it is possible to check which oligonucleotides are positive by hybridization, feed the results into a computer and use a program to look for sequence overlaps in order to establish the required DNA sequence. DNA microarrays have permitted sequencing by hybridization to oligonucleotides on a large scale.

Single strand conformation analysis can also be used to determine if the purified and isolated DNA from a subject has particular allele, haplotype or SNP. The conformation of the single-stranded DNA can alter based upon a single base change in the sequence, causing the DNA to migrate differently on electrophoresis. The analysis can involve four steps: (1) polymerase chain reaction (PCR) amplification of DNA sequence of interest; (2) denaturation of double-stranded PCR products; (3) cooling of the denatured DNA (single-stranded) to maximize self-annealing; and (4) detection of mobility difference of the single-stranded DNAs by electrophoresis under non-denaturing conditions. Additionally, the SSCP mobility shifts must be visualized which is done by the incorporation of radioisotope labeling, silver staining, fluorescent dye-labeled PCR primers, and more recently, capillary-based electrophoresis.

### Kits

It is contemplated that all of the diagnostic and screening assays disclosed herein can be in kit form for use by a health care provider and/or a diagnostic laboratory.

For example, for Aβ and sAPPα and sAPPβ peptide testing, such a kit could include antibodies that recognize the peptide of interest, reagents for isolating and/or purifying protein from a biological tissue or bodily fluid, reagents for performing assays on the isolated and purified protein, instructions for use, and reference values or the means for obtaining reference values for the quantity or level of peptides in a control sample.

In a preferred embodiment, antibodies that recognize Aβ40, Aβ42, sAPPα and sAPPβ would be included in one kit so that assays for all of the peptides related to amyloid could be performed.

Diagnostic and screening assays based upon nucleotide testing can also be incorporated into kits. For example, probes and/or primers for each of the Apo E alleles, reagents for isolating and purifying nucleic acids from biological tissue or bodily fluid, reagents for performing assays on the isolated and purified nucleic acid, instructions for use, and comparison sequences could be included in a kit for detection of the Apo E alleles. A further embodiment would be a kit with all the components for testing of the Apo E alleles and the peptides related to amyloid.

Kits for screening and diagnosis utilizing the H1 haplotype of the MAPT locus are also contemplated by the invention. These kits could include probe and/or primers specific for the H1 haplotype, reagents for isolating and purifying nucleic acids from biological tissue or bodily fluid, reagents for performing assays on the isolated and purified nucleic acid, instructions for use, and comparison sequences could be included in a kit for detection of the H1 haplotype.

Kits for screening and diagnosis utilizing the polymorphisms designated rs35134565 and rs5820605 are also contemplated by the invention. These kits could include probe and/or primers specific for the polymorphisms, reagents for isolating and purifying nucleic acids from biological tissue or bodily fluid, reagents for performing assays on the isolated and purified nucleic acid, instructions for use, and comparison sequences could be included in a kit for detection of the polymorphisms.

A preferred embodiment is a kit including components for testing for both the H1 haplotype and the polymorphisms.

### Drug Screening Assays and Research Tools

All of the biomarkers disclosed herein can be used as the basis for drug screening assays and research tools.

In the simplest models, sAPPα and sAPPβ peptide can be used in drug screening assays. The peptides can be used in drug screening tests free in solution or affixed to a solid support. Procaryotic or eukaryotic host cells transformed with nucleotides that express sAPPα or sAPPβ peptides can also be used. All of these forms can be used in binding assays to determine if agents being tested form complexes with the peptides.

Thus, a further embodiment of the present invention is a method for screening for drugs comprising contacting an agent to be tested with a sAPPα and/or sAPPβ peptide and assaying for the presence of complexes between the peptide and the agent by methods known in the art.

High throughput screening can also be used to screen for drugs. Small peptides or molecules can be synthesized and bound to a surface and contacted with the sAPPα and/or sAPPβ peptide and washed. The bound peptide is visualized and detected by methods known in the art.

Antibodies to the peptides can also be used in competitive drug screening assays. The antibodies compete with the agent being tested for binding to the peptides. The antibodies can be used to find agents that have antigenic determinants on the peptides.

The nucleotide markers can also be used in various drug screening assays and as research tools.

Host cells can be transformed with DNA comprising the Apo E alleles, the H1 haplotype the MAPT gene, the H1 haplotype of the 3′ UTR, or the polymorphisms designated rs5820605 and rs35234656 by methods known in the art.

The resulting transformed cells can be used for testing for therapeutic agents. Specifically, cells can be transformed with any one of the ApoE alleles, ε2, ε3, or ε4, and contacted with a potential therapeutic agent. The resulting expression of the allele can be detected and compared to the expression of the allele in the cell before contact with the agent.

The expression of the alleles in host cells can be detected and measured by any method known in the art, including but not limited to, luciferase reporter gene assay.

Host cell can also be transformed with the H1 3′UTR as well as the H1 3′UTR with and without the polymorphisms designated rs5820605 and rs35234656. Such a method is exemplified in Example 7 using a luciferase report gene assay. As shown in this Example, these cells can also be contacted with potential therapeutic agents and the expression of the inserted DNA detected and measured before and after contact with the agent.

These transformed host cells can also be used for further research. For instance, as shown in Example 7, these constructs can be contacted with peptides and other substances naturally occurring in both healthy controls and patients with AD and TPD to see the effects on the gene expression.

The H1 3′UTR can also be linked to other cells with measurable phenotypes such as tau. Expression of the gene linked to the H1 3′UTR can be measured before and after the contact with a potential therapeutic agent, as well as a naturally occurring peptide or molecule.

These gene constructs as well as the host cells transformed with these gene constructs can also be the basis for transgenic animals for testing both as research tools and for therapeutic agents. Such animals would include but are not limited to, nude mice and drosophila. Phenotypes can be correlated to the genes and looked at in order to determine the genes effect on the animals as well as the change in phenotype after administration or contact with a potential therapeutic agent.

### EXAMPLES

The present invention may be better understood by reference to the following non-limiting examples, which are presented in order to more fully illustrate the preferred embodiments of the invention. They should in no way be construed to limit the broad scope of the invention.

### Example 1

### Patient Samples and Statistical Analysis

Autopsy brain samples were obtained from seven centers (Table 1). The primary source of material was the brain bank at Columbia University Medical Center (CUMC) (New York, N.Y.) (Tables 2 and 3). Secondary sources were the University of California San Diego (San Diego, Calif.), the University of Kentucky (Lexington, Ky.), the Banner Health Banner Sun Health Research Institute (Sun City, Ariz.), Northwestern University (Chicago, Ill.), the University of Washington (Seattle, Wash.) and Washington University (St. Louis, Mo.). Patient data for each component of this study are summarized in Table 4. Neuropathological examination was per the protocols of the respective institutions.

Inclusion criteria for TPD were: 1) frequent NFT corresponding to Braak NFT stage III-IV (Braak et al. (1993)) and no or very rare NFT in the frontal, parietal or occipital cortex; 2) no more than sparse amyloid plaques (CERAD (Mina et al. (1991)) score 0 or A); and 3) no other neuropathological substrate for dementia. All TPD cases had been clinically classified pre-mortem as either possible or probable AD (n=31) or mild-cognitive impairment (n=3) by their respective source institutions. For APOE genotype comparisons, neuropathologically-confirmed AD patients aged 75 years or higher from the CUMC cohort categorized as CERAD plaque score of C and Braak NFT stage of V-VI were used. Successful cerebral aging was defined as: 1) age greater than or equal to 80 years; 2) CERAD plaque score of 0; and 3) Braak NFT stage of 0-II. All subjects were of Caucasian ancestry.

For the statistical analysis for ELISA, quantitative immunoblotting, luciferase assays, and QPCR experiments, the statistical significance was determined by one-way ANOVA and Tukey test or Student's t-test in GraphPad Prism (GraphPad Software, La Jolla, Calif.). Statistical outliers and specimens with measurement errors were excluded.

For statistical analysis for APOE comparisons, a Fisher's Exact Test performed in Microsoft Excel was used.

For MAPT haplotype comparisons and single locus associations, a Chi-squared test was performed using plink (Purcell et al. (2007).

TABLE 1
Summary of Patient Data from All Seven Centers

| | | | | Average | | CERAD | |
|---|---|---|---|---|---|---|---|
| Classi- | | | | Age yr. | Braak | Plaque | Clin. |
| fication | n | Male | Female | (Range) | NFT | Score | Diag. |
| Control | 56 | 23 | 33 | 86.9 (67-108) | 0-11 | 0-A | normal |
| TPD | 34 | 11 | 23 | 90.2 (65-103) | III-IV | 0-A | AD or |
| | | | | | | | MCI |
| AD | 50 | 19 | 31 | 86.7 (73-104) | V-VI | B-C | AD |

TABLE 2
Clinical Patient Data for Patients from CUMC

| | | | | | | Time since |
|---|---|---|---|---|---|---|
| Study | | | Clinical | | Last | MMSE |
| case No. | Age | Sex | dementia | CDR | MMSE | (yr) |
| 1 | 65 | F | DEM | 0.5 | 22 | 1 |
| 2 | 76 | F | NA | NA | NA | NA |
| 3 | 80 | M | CI | NA | 22 | 5 |
| 4 | 85 | M | DEM | 1 | NA | NA |
| 5 | 86 | F | DEM | NA | 29 | 1 |
| 6 | 86 | M | DEM | NA | NA | NA |
| 7 | 90 | F | CI | 0.5 | NA | NA |
| 8 | 91 | F | DEM | 0.5 | 21 | 1 |
| 9 | 92 | F | DEM | NA | NA | NA |
| 10 | 93 | F | DEM | NA | 5 | 4 |
| 11 | 94 | F | DEM | NA | 26 | 1 |
| 12 | 95 | F | DEM | 1 | NA | NA |
| 13 | 97 | F | CI | NA | NA | NA |

TABLE 3
Neuropathological Data of Patients From CUMC

| | | | CERAD age- | |
|---|---|---|---|---|
| | | Braak | related plaque | PMI |
| Study case No. | Classification | NFT | score | (min) |
| 1 | TPD | III-IV | 0 | 275 |
| 2 | TPD | III-IV | 0 | NA |
| 3 | TPD | III-IV | 0 | 286 |
| 4 | TPD | III-IV | 0 | 1065 |
| 5 | TPD | III-IV | 0 | NA |
| 6 | TPD | III-IV | 0 | NA |
| 7 | TPD | III-IV | 0 | 325 |
| 8 | TPD | III-IV | 0 | 117 |
| 9 | TPD | III-IV | 0 | 705 |
| 10 | TPD | III-IV | 0 | 120 |
| 11 | TPD | III-IV | 0 | 280 |
| 12 | TPD | III-IV | 0 | 110 |
| 13 | TPD | III-IV | 0 | 205 |

TABLE 4
Summary of Patient Data from Each Study

| | Control | TPD | AD |
|---|---|---|---|
| Ab ELISA - frontal cortex | | | |
| n (male/female) | 16 (8/8) | 11 (2/9) | 8 (4/4) |
| Average PMI (Standard deviation) | 126 (111) | 496 (541) | 388 (128) |
| Age at death (range) | 86.4 (74-94) | 88.8 (65-103) | 85 (73-98) |
| Ab ELISA - hippocampus | | | |
| n (male/female) | 6 (4/2) | 6 (1/5) | 5 (2/3) |
| Average PMI (Standard deviation) | 202 (823) | 783 (534) | 560 (355) |
| Age at death (range) | 79.8 (67-92) | 92.7 (85-103) | 91.0 (84-98) |
| APP/sAPP immunoblot - Frontal Cortex | | | |
| n (male/female) | 11 (5/6) | 13 (3/10) | — |
| Average PMI (Standard deviation) | 281 (255) | 422 (505) | — |
| Age at death (range) | 87.1 (74-97) | 86.8 (65-96) | — |
| QPCR | | | |
| n (male/female) | 9 (4/5) | 9 (2/4) | 6 (2/4) |
| Average age at death in yr (range) | 87 (55-97) | 89 (82-97) | 93 (86-102) |
| Average RNA-integrity number | 6.1 | 5.8 | 4.0 |
| Tau sarkosyl immunoblots - Hippocampus | | | |
| n (male/female) | 5 (3/2) | 6 (2/4) | 5 (3/2) |
| Average PMI in min (standard deviation) | 668 (907) | 381 (393) | 558 (534) |
| Average age at death in yr (range) | 80 (67-92) | 93 (85-103) | 91 (84-96) |
| Tau immunoblots - Hippocampus | | | |
| n (male/female) | 5 (3/2) | 5 (0/5) | 4 (2/2) |
| Average PMI in min (standard deviation) | 609 (941) | 221 (96) | 662 (556) |
| Average age at death in yr (range) | 80 (67-92) | 94 (90-103) | 89 (84-96) |
| Tau immunoblots - Frontal cortex | | | |
| n (male/female) | 12 (6/6) | 13 (3/10) | — |
| Average PMI in min (standard deviation) | 326 (321) | 422 (505) | — |
| Average age at death in yr (range) | 88 (65-94) | 86.8 (65-96) | — |
| MAPT resequencing | | | |
| n (male/female) | 5 (3/2) | 10 (9/1) | — |
| Average age at death in yr (range) | 90 (85-93) | 89 (65-97) | — |
| MAPT association analysis | | | |
| n (male/female) | 48 (18/30) | 34 (11/23) | — |
| Average age at death in yr (range) | 88.1 (78-108) | 90.2 (82-100) | — |
| APOE association analysis | | | |
| n (male/female) | 56 (27/29) | 32 (10/22) | 50 (19/31) |
| Average age at death in yr (range) | 83.3 (80-100) | 90.4 (81-100) | 86.7 (75-104) |

### Example 2

### Neuropathological Analysis

Materials and Methods

Patient material from Columbia University was subjected to a detailed neuropathological analysis (Tables 2 and 3).

Immunohistochemistry was performed on 6 μm paraffin-embedded sections as previously described (Crary et al. (2006)), using various antisera found in Table 5.

For transmission electron microscopy, a portion of CA1, previously fixed in 10% neutral-buffered formalin, was post-fixed with 2.5% glutaraldehyde in 0.1 M Sorenson's buffer (pH 7.2) followed by 1% OsO4 in Sorenson's buffer for one hour and embedded in Lx-112 (Ladd Research Industries, Inc.). 60 nm sections were stained with uranyl acetate and lead citrate and examined under a JEOL JEM-1200 EXII electron microscope and imaged using an ORCA-HR digital camera (Hamamatsu Photonics, Japan).

Results

A consecutive brain autopsy series of 992 patients performed at Columbia University Medical Center was retrospectively reviewed. Of the 336 patients clinically classified as possible or probable AD (McKhann et al. (1984)), 13 meet the neuropathological criteria for TPD (Yamada (2003)). This represents 3.8% of all dementia patients, but increases to 7.3% of patients 90 years or above. There is a female preponderance and an average age of death of 86.9 years (range 65-97 years), which is older than the average for AD patients in this series (74 years).

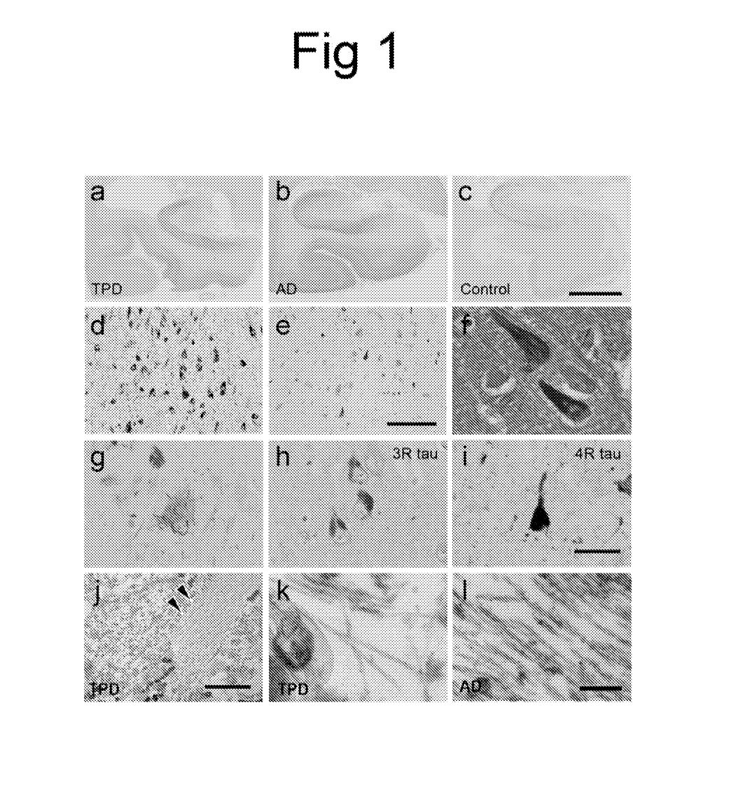

Post-mortem examination of these 13 TPD patients reveals a pattern reminiscent of early to moderate-stage AD. There is gross medial temporal lobe atrophy compared to controls. Unlike most late-stage AD patients, frontal, parietal and occipital cortices are preserved in TPD. Microscopically, TPD brains exhibit severe medial temporal lobe tauopathy with frequent NFT (FIGS. 1a, 1d-g). All these cases exhibit numerous extracellular (“ghost”) tangles (FIG. 1g), associated with abnormal degenerating argyrophilic neurites.

Consistent with previous studies, NFT in TPD are immunopositive with specific antisera to 3R and 4R tau (FIGS. 1h and i), as well as for various phospho-tau specific epitopes shown in Table 6, which is the same profile as seen in AD and certain rare tauopathies (Ikeda et al. (1999); Iseki et al. (1997); Noda et al. (2006)). Moreover, it was confirmed that extracellular NFT have disproportionate immunolabeling for 3R tau in TPD, as previously reported (Iseki et al. (2006)).

To determine whether there are unique ultrastructural features in TPD, electron microscopy was performed in the cornu ammonis 1 (CA1) sector of the hippocampal formation. Examination of epoxy resin ultrathin sections shows filaments that are suggestive of paired-helical filaments (PRFs) in TPD (n=4), as is observed in AD (FIGS. 1j-l) (Kidd (1963)). Ultrathin sections were also evaluated for insoluble amyloid deposits and inclusions, but none were observed. Aside from NFT, there are no ubiquitin-positive inclusions and no more than incidental α-synuclein-positive inclusions in the locus coeruleus, pars compacta of the substantia nigra, hypothalamus and substantia innominata in two patients. Vascular disease is frequent, as is common in aging, but there was no ischemic injury sufficient in magnitude or distribution, to cause dementia.

TABLE 5
Antisera

| Antigen | | | | | |
|---|---|---|---|---|---|
| (clone) | Dilution | Type | Epitope | Notes | Source |
| APP | 1:4000 | Mouse | N-term | Detects full- | Millipore |
| (22C11) | | Monoclonal | | length and | |
| | | | | secretory | |
| | | | | fragments | |
| APP/Aβ | 1:1000 | Mouse | AA 1-16 of | Detects APP | Covance |
| (6E10) | | Monoclonal | b-amyloid | and Ab | |
| sAPPa | 1:1000 | Mouse | Neo-epitope | No cross | American |
| (2B3) | | Monoclonal | after a- | reactivity with | Research |
| | | | secretase | full-length APP | Products |
| | | | cleavage | | |
| sAPPb | 1:500 | Rabbit | Neo-epitope | No cross | Covance |
| | | Polyclonal | after b- | reactivity with | |
| | | | secretase | full-length APP | |
| | | | cleavage | | |
| Tau (HT7) | 1:1000 | Mouse | — | Total tau | Thermo Scientific |
| | | Monoclonal | | | |
| Tau (RD3, | 1:1000 | Mouse | 209-224 | 3R specific | Millipore |
| 8E6/C11) | | Monoclonal | | | |
| Tau (RD4, | 1:500 | Mouse | 275-291 | 4R specific | Millipore |
| 1E1/A6) | | Monoclonal | | | |
| Tau (AT8) | 1:500 | Mouse | S202/T205 | Late epitope | Thermo Scientific |
| | | Monoclonal | | | |
| Tau (MC6) | 1:500 | Mouse | S235 | — | Dr. Peter Davies |
| | | Monoclonal | | | |
| Tau (CP9) | 1:500 | Mouse | T231 | Affects MT | Dr. Peter Davies |
| | | Monoclonal | | binding, early | |
| | | | | epitope | |
| Tau (TG3) | 1:500 | Mouse | T231 | Conformation | Dr. Peter Davies |
| | | Monoclonal | | specific | |
| Tau (PG5) | 1:500 | Mouse | S409 | — | Dr. Peter Davies |
| | | Monoclonal | | | |
| Tau (CP13) | 1:500 | Mouse | S202 | — | Dr. Peter Davies |
| | | Monoclonal | | | |
| Tau (CP3) | 1:500 | Mouse | S214 | — | Dr. Peter Davies |
| | | Monoclonal | | | |
| GAPDH | 1:2500 | Mouse | — | — | Millipore |
| | | Monoclonal | | | |
| Ubitquitin | 1:300 | Mouse | — | — | DakoCytomation |
| | | Monoclonal | | | |
| a-synuclein | 1:50 | Mouse | — | — | Leica |
| (UKKM51) | | Monoclonal | | | |
| TDP-43 | 1:2000 | Mouse | — | — | ProteinTech |
| | | Monoclonal | | | Group |

TABLE 6
Analysis of phospho-tau in paraffin sections from TOD by
immunohistochemistry

| | | | | | | | Temporal | | | | | |
|---|---|---|---|---|---|---|---|---|---|---|---|---|
| | Hippocampus | | | | | | cortex | | | Frontal cortex | | |
| | (CA1) | | | Amygdala | | | (BA38) | | | (BA9) | | |
| Antisera | TPD | AD | Control | TPD | AD | Control | TPD | AD | Control | TPD | AD | Control |
| AT8 (pS202/pT205) | + | + | − | + | + | +/− | + | + | +/− | − | + | − |
| MC6 (pS235) | + | + | +/− | + | + | +/− | + | + | +/− | − | + | − |
| CP9 (pT231) | + | + | +/− | + | + | +/− | + | + | +/− | − | + | − |
| TG3 (pT231) | + | + | +/− | + | + | +/− | + | + | +/− | − | + | − |
| PG5 (pS409) | + | + | +/− | + | + | +/− | + | + | +/− | − | + | − |
| CP13 (pS202) | + | + | +/− | + | + | +/− | + | + | +/− | − | + | − |
| CP3 (pS214) | + | + | +/− | + | + | +/− | + | + | +/− | − | + | − |

+ = frequent
+/− = scattered,
− = negative

### Example 3

### Biochemical Analysis of Tau

Materials and Methods

For biochemical analysis of tau, protein extracts were prepared from the Brodmann area 9 (BA9) of the frontal lobe, and CA1, of fresh-frozen human brain from patients in the expanded cohort described in Example 1 as described by Takahashi et al. (2002) with modifications. Briefly, fresh-frozen brain tissue was homogenized using 10 volumes (wt/vol) of extraction buffer containing 20 mM (4-[2-hydroxyethyl]-1-piperazineethanesulfonic acid]) (HEPES), pH 7.4, 100 mM NaCl, 20 mM NaF, 1% Triton X-100, 1 mM sodium orthovanadate, 5 mM EDTA containing a Complete Mini protease inhibitor cocktail (Roche Applied Science, Indianapolis, Ind.) supplemented with 2 mM PMSF using 15 strokes with a Teflon-coated pestle. Homogenates were centrifuged at 3000×g at 4° C. for 5 minutes. The supernatant (crude total tau fraction) was aliquoted and stored at −80° C.

Preparation of sarkosyl-soluble and insoluble fractions was carried out as described Ksiezak-Ridings and Wall (1994). Briefly, homogenates were centrifuged at 27,000×g for 20 minutes. The pellet was resuspended in buffer containing 0.8 M NaCl and 10% sucrose at 10 ml/g of initial tissue and recentrifuged at 27,000×g for 20 minutes. The supernatant was incubated in the presence of 1% sarkosyl for 1 hour at 25° C. Finally, the sample was centrifuged at 100,000×g for 2 hours. The resulting pellet was considered the sarkosyl-insoluble tau fraction.

Samples were resolved by 10% SDS-PAGE, and analyzed by immunoblot with various antisera (Table 5). Some sarkosyl-insoluble fractions were applied to grids, allowed to dry for 20 minutes and negatively stained with 1% lithium phosphotungstate and examined by electron microscopy in Example 2.

Results

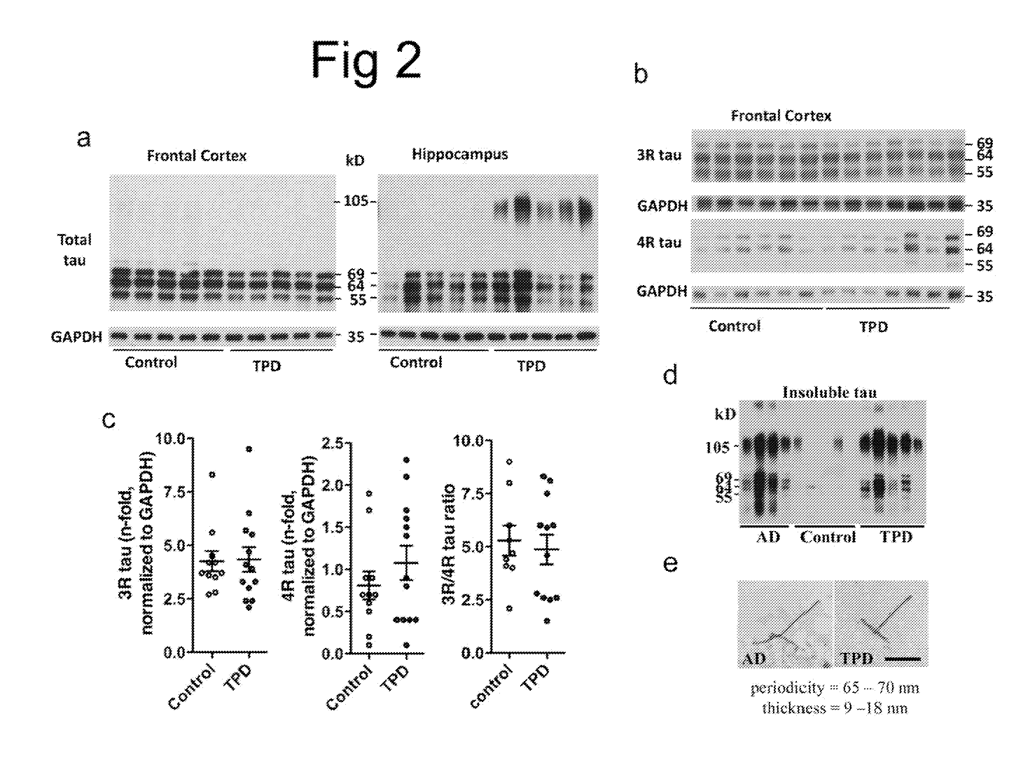

To determine whether differences in tau isoform expression occur in TPD, a biochemical analysis was performed in the Brodmann area 9 (BA9) region of the frontal cortex and CA1 region of the hippocampus using an expanded cohort that included specimens from multiple AD research centers in the United States. In both TPD (n=13) and control (n=12) subjects, immunoblots using total protein from BA9 and probed for tau reveal the three major bands at 55, 64 and 69 kD (FIG. 2a). In CA1 of the TPD patient, an additional high-molecular weight band is present at about 105 kD, representing tau aggregates.

Immunoblots using antisera specifically recognizing 3R and 4R tau show no difference in the levels or ratio of tau isoforms between TPD (n=13) and control (n=11; FIGS. 2b and 2c). Protein fractions enriched for insoluble tau isolated by sarkosyl extraction from CA1 and examined by immunoblot reveal no difference in the tau isoform expression in TPD (n=5) and in AD (n=6) (FIG. 2d).

Ultrastructural studies of sarkosyl fractions confirm the presence of PHFs in TPD as found in AD (FIG. 2e).

Taken together, the data show that the NFTs in TPD are regionally, histologically, biochemically and ultrastructurally similar to those in early to moderate-stage AD.

### Example 4

### Biochemical Characterization of Aβ and APP in TPD

Materials and Methods For biochemical analysis of Aβ, APP and sAPPα/β, preparation of protein extracts was performed as described by Schmidt et al. (2005) with modifications. Fresh-frozen autopsy brain tissue from patients in Example 1, was homogenized with 10 volumes (wt/vol) of buffer [250 mM sucrose, 20 mM Tris-HCl (pH 7.4), 1 mM ethylenediaminetetraacetic acid (EDTA), 1 mM ethylene glycol tetraacetic acid (EGTA)] containing a Complete Mini protease inhibitor cocktail (Roche Applied Science, Indianapolis, Ind.) supplemented with 1 mM phenylmethylsulfonyl fluoride (PMSF) using 20 strokes with a Teflon-coated pestle, aliquoted and frozen at −80° C. Protein concentration was measured by bicinchoninic acid protein assay (Pierce, Rockford, Ill.). For extraction of soluble Aβ, homogenate was combined with an equal volume of 0.4% diethylamine (DEA) in 100 mM NaCl and centrifuged at 100,000×g for 1 hr at 4° C. Then, the supernatant was combined with an equal volume of 0.5 M Tris base, pH 6.8.

ELISAs were performed using Aβ x-40 and x-42 BetaMark chemiluminescent kits (Covance Inc., Princeton, N.J.).

For immunoblotting, the homogenates were separated into membrane (pellet) and cytosolic (supernatant) fractions by centrifugation at 100,000×g for 1 hour at 4° C. The pellet was resuspended in homogenization buffer, subjected to SDS-PAGE, transferred to nitrocellulose and probed with various antisera (Table 5) and visualized by chemiluminescence. Densitometric analysis was performed using NIH Image J.

Results

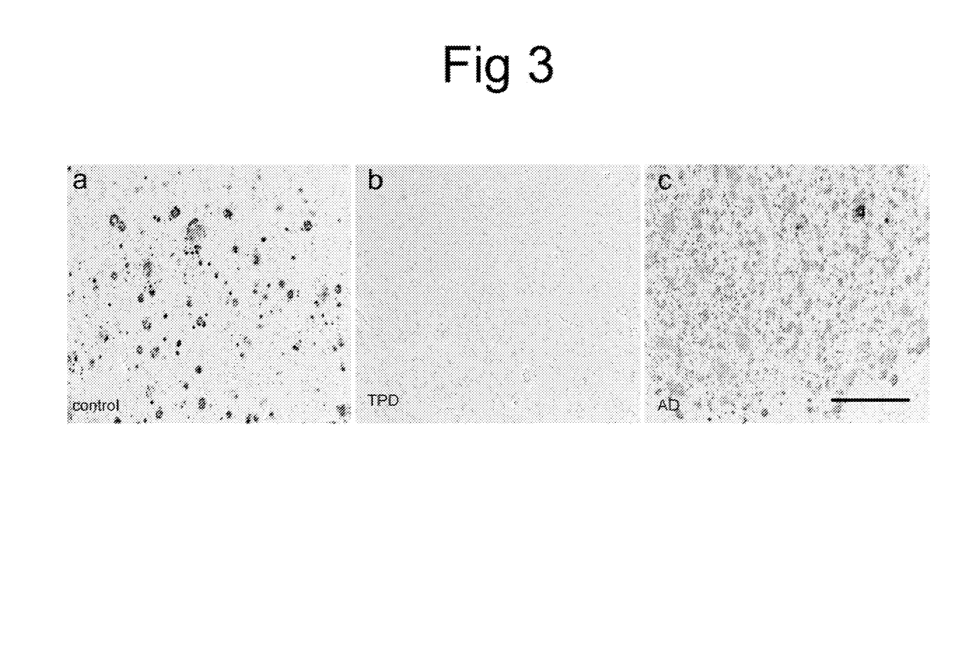

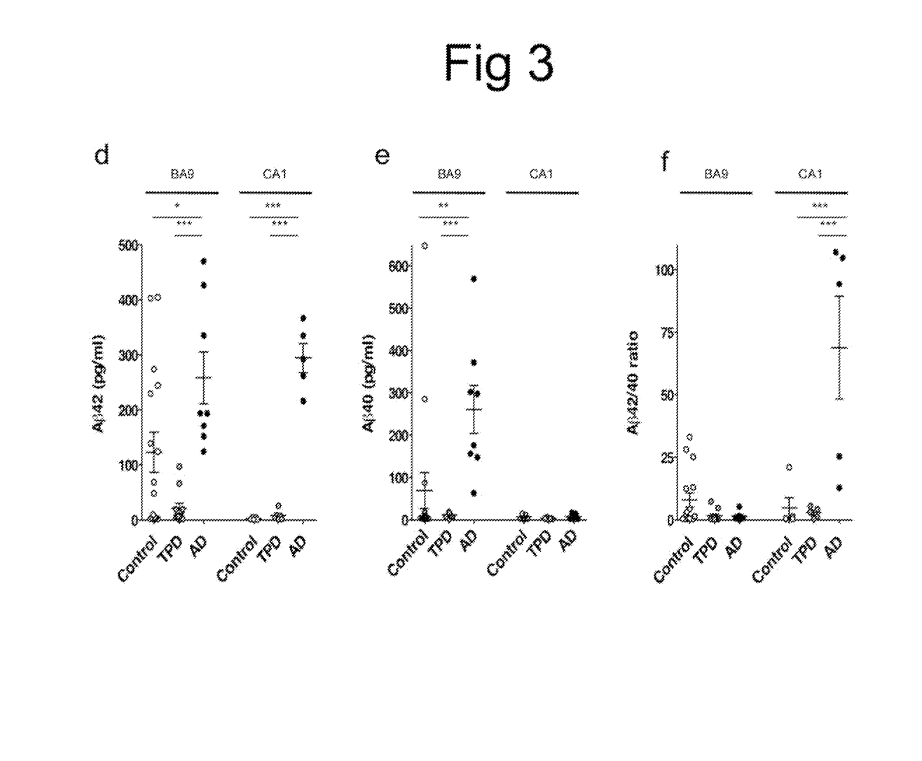

While, immunohistochemistry shows no significant Aβ deposition in TPD (FIGS. 3a-c), soluble Aβ is histologically invisible. ELISAs show significantly lower soluble Aβ42 levels in TPD (n=11, p<0.001) and controls (n=16, p<0.05) as compared to AD (n=8) in BA9 (FIG. 3d). ELISAs also show significantly lower levels of soluble Aβ42 in TPD (n=6, p <0.001) and controls (n=5, p<0.001) compared to AD (n=5) in CA1, a region undergoing neurodegeneration in TPD (FIG. 3d). Significant soluble Aβ42 levels are lower in TPD when compared to control subjects in BA9. Measurements of the less fibrillogenic Aβ40 species (Jarrett et al. (1993)) reveal significantly lower soluble Aβ40 levels in TPD (p<0.001) and control (p<0.01) than AD in BA9 but not CA1 (FIG. 3e). There is a higher Aβ42/40 ratio in AD in CA1 compared to TPD (p<0.001) and control (p<0.001), but there is no difference between TPD and control (FIG. 3f).

These findings show that TPD brain parenchyma has low levels of soluble Aβ when compared to AD. The control brains have variable levels of soluble Aβ that overlap with those observed in AD and TPD.

Low Aβ may arise from decreased production, decreased fibrillization or increased clearance. Quantitative immunoblots reveal a significantly reduced level of the full length APP holoprotein in TPD (n=14) compared to controls (n=11, p<0.001) in BA9 (FIGS. 4a and b). Decreased levels of full-length APP in AD as well were observed, consistent with previous reports (Davidisson et al. (2001); Wu et al. (2011)). TPD is unlike AD in that BA9 is preserved, leading to the conclusions that low APP levels in TPD reflect differences in underlying APP metabolism rather than neuronal loss and gliosis.

Using specific antisera recognizing neoepitopes formed by secretase cleavage, significantly lower sAPPβ levels were found (p<0.001), and significantly higher sAPPα (p<0.05) (n=14), as compared to controls (n=11) in BA9.

Finally, there is no difference in the levels of APP mRNA among TPD (n=8), AD (n=6) and control (n=9) in BA9 (FIG. 4c), suggesting that non-amyloidogenic processing contributes to decreased production of Aβ in TPD.

Apolipoprotein E (ApoE) alleles correlate with AD risk and amyloid plaque load (Corder et al. (2006); Saunders et al. (1993)). The ApoE allele frequency was determined and found both a significant decrease in ε4 (p=5.78 x 10-7) in TPD (n=32) compared to age-matched AD (n=50) and an increase in ε2 (p=5.58 x 10-5) and ε3 (p=0.037), (Table 7), which is consistent with previous studies (Ikeda et al. (1999); Jellinger and Bancher (1998)). Compared to controls (n=56), there are decreased ε4 and increased ε2 frequencies in TPD, but these differences are not significant. These data support the well-established finding that ApoE ε4 is associated with Aβ deposition and ε2 is protective (Corder et al. (1993)).

TABLE 7
APOE Genotypes

| | | | | | | p v TPD | | | |
|---|---|---|---|---|---|---|---|---|---|
| | Allele | | | p v TPD (e2) | | (e3) | | p v TPD (e4) | |
| | count | | | one- | two- | one- | two- | one- | two- |
| | e2 (%) | e3 (%) | e4 (%) | tailed | tailed | tailed | tailed | tailed | tailed |
| TPD | 12 (19) | 49 (77) | 3 (4) | | | | | | |
| Control | 12 (11) | 82 (77) | 14 (13) | 0.12 | 0.18 | 0.54 | 1.00 | 0.063 | 0.112 |
| AD | 1 (1) | 62 (62) | 37 (37) | 5.58E−05 | 5.58E−05 | 0.037 | 0.061 | 5.78E−07 | 1.43E−06 |

significant p values are in bold (Fischer's exact test)

### Example 5

### Genetic Analysis of MAPT in TPD

Materials and Methods

Resequencing and Association Analysis

For genomic DNA isolation, fresh-frozen brain was lysed overnight at 55° C. under continuous rotation in 500 μl of buffer [4 M urea, 10 mM of EDTA, 0.5% sarkosyl, 0.1 M Tris HCl pH (8.0), 0.1 M NaCl and 20 mg/ml proteinase K]. DNA was purified by phenol-chloroform extraction. MAPT target enrichment was performed with the RDT1000 system (RainDance Technologies, Lexington Mass.) using 464 primer pairs spanning greater than 99.9% of MAPT totaling 140,252 bp (hg18; chr17: 41,324,942-41,465,194) and 243,995 bp of amplicons designed using Primer3 software. Amplicons were sequenced on the 454 platform (Roche 454 Life Sciences, Branford, Conn.). Performance was analyzed using CLC Genomics Workbench (CLC bio, Cambridge, Mass.; Table 8).

Variants were identified with gsMapper (Roche). Variants were called if identified on three or more reads, with a total read coverage of six, and counted only if they were observed on forward and reverse reads. Variants on 10-90% of reads were called as heterozygous and those on greater than 90% homozygous. Allele counts and frequencies were calculated and used to generate p values (Fisher's exact test). Variants meeting one the following criteria (after filtering for H2 haplotype-tagging variants) were included for validation: 1) in coding regions or untranslated regions; 2) TPD specific and control specific; 3) p<0.025 (Fisher's exact test, allelic, unadjusted). Validation and genotyping of variants was performed on a Sequenom MassArray iPlex platform (Sequenom, San Diego, Calif.) or Sanger sequencing. Subjects were also genotyped for APOE status using the rs7412 and rs429358 polymorphisms (Ghebranious et al. (2005); Clark et al. (2009)).

Results

Using a set of markers previously employed to tag MAPT haplotype diversity (Pittman et al. (2005)), there is no observed difference in the common H1 subhaplotypes, including H1c, between TPD (n=34) and controls (n=48). However, there is a difference in the H2 frequency in TPD compared to controls (p=0.015) (Table 9), but this finding is not significant following Bonferroni correction (p=0.075).

Post-hoc analysis reveals that the trend towards significance is derived from differences between TPD patients and a subset of controls. Neurodegeneration is common in elderly individuals classified as cognitively normal (Bouros et al. (1994); Price and Morris (1999)). Employing a model proposed by Rowe and Kahn (1987), a subset of the oldest-old controls was identified (average age=89.3 years) that are exceptional based on their mild NFT burden and absence of amyloid plaques. This control group, termed “successful cerebral aging”, represented the ultimate in healthy brain aging. When successful cerebral aging controls (n=28) are compared directly to TPD (Table 10), the difference in H1 allele frequency is highly significant (p=0.004), even after adjusting for multiple comparisons (p=0.022). These results are consistent with the hypothesis that the H1 haplotype is a risk factor for limbic NFT formation in TPD and that H2 is protective.

TABLE 8
MAPT resequencing summary statistics

| | | | | | Mean | | | | | |
|---|---|---|---|---|---|---|---|---|---|---|
| Sample | | Mapped | Target | | Base | | | | | Base |
| ID | Classification | Reads | Reads | Specificity | Coverage | C1 | C10 | C20 | C100 | coverage |
| 1 | Control | 33,448 | 26,763 | 80.0% | 68 | 99.4% | 95.6% | 87.9% | 20.8% | 93.3% |
| 2 | Control | 49,530 | 30,143 | 60.9% | 78 | 99.5% | 96.4% | 90.3% | 26.1% | 93.6% |
| 3 | Control | 52,629 | 46,781 | 88.9% | 132 | 99.3% | 98.5% | 96.9% | 61.8% | 95.6% |
| 4 | Control | 53,118 | 33,749 | 63.5% | 88 | 99.5% | 97.1% | 91.7% | 33.2% | 93.6% |
| 5 | Control | 81,592 | 66,067 | 81.0% | 186 | 99.5% | 98.9% | 98.1% | 78.3% | 95.4% |
| 6 | TOD | 77,646 | 60,200 | 77.5% | 166 | 99.5% | 99.1% | 98.1% | 74.4% | 96.1% |
| 7 | TOD | 121,761 | 66,311 | 54.5% | 161 | 99.6% | 98.4% | 96.6% | 64.1% | 92.7% |
| 8 | TOD | 48,950 | 37,029 | 75.6% | 98 | 99.5% | 97.2% | 92.4% | 40.0% | 93.0% |
| 9 | TOD | 62,833 | 31,373 | 49.9% | 83 | 99.5% | 97.4% | 92.2% | 31.9% | 94.6% |
| 10 | TOD | 27,213 | 18,812 | 69.1% | 50 | 99.1% | 93.5% | 82.6% | 5.8% | 94.6% |
| 11 | TOD | 48,171 | 27,617 | 57.3% | 77 | 99.5% | 95.8% | 90.3% | 28.4% | 93.1% |
| 12 | TOD | 71,422 | 55,360 | 77.5% | 152 | 99.6% | 98.9% | 97.5% | 70.1% | 95.6% |
| 13 | TOD | 45,344 | 29,557 | 65.2% | 76 | 99.4% | 95.3% | 88.3% | 25.3% | 91.7% |
| 14 | TOD | 46,755 | 31,207 | 66.7% | 76 | 99.4% | 94.7% | 88.5% | 25.8% | 91.3% |
| 15 | TOD | 80,843 | 60,510 | 74.8% | 149 | 99.5% | 98.6% | 96.8% | 61.9% | 93.8% |
| | Average | 60,084 | 41,432 | 69.5% | 109 | 99.5% | 97.0% | 92.5% | 43.2% | 93.9% |

TOD = tangle-only dementia;
Mapped reads: total number of reads mapping to the human genome;
target reads: mapped reads that include the target;
mean base coverage: average base coverage within target. The target includes all amplicon sequences, with primer sequences excluded.
C1: % of target that has at least 1x base coverage. Note, non-unique sequencing reads are mapped randomly.
C20: % of target that has at least 20x base coverage.
C100: % of target that has at least 100x base coverage.
Base coverage (0.2x of mean): % of target that has at least 20% of mean base coverage.

TABLE 9
Association of common MAPT haplotypes with TPD v. Control

| | | TPD | | | | |
|---|---|---|---|---|---|---|
| Hap- | | (n = 34) | v. Control | | | |
| lo- | | Fre- | (n = 48) | | | |
| type | Allele | quency | Frequency | CHISQ | P | Corrected |
| H2a | AGGCCG | 0.21 | 0.39 | 5.921 | 0.0015 | 0.075 |
| H1b | GGGCTA | 0.12 | 0.1 | 0.165 | 0.685 | 1 |
| H1c | AAGTTG | 0.16 | 0.09 | 1.453 | 0.228 | 1 |
| H1d | AAGCTA | 0.04 | 0.05 | 0.195 | 0.659 | 1 |
| H1e | AGGCTA | 0.15 | 0.1 | 1.193 | 0.275 | 1 |

TABLE 10
Association of common MAPT haplotypes with TPD v. Successful
Cerebral Aging

| | | | v. | | | |
|---|---|---|---|---|---|---|
| | | | Successful | | | |
| | | | Cerebral | | | |
| | | TPD | Aging | | | |
| Haplo- | | (n = 34) | (n = 28) | | | |
| type | Allele | Frequency | Frequency | CHISQ | P | Corrected |
| H2a | AGGCCG | 0.21 | 0.45 | 8.077 | 0.004 | 0.022 |
| H1b | GGGCTA | 0.12 | 0.09 | 0.356 | 0.551 | 1 |
| H1c | AAGTTG | 0.16 | 0.11 | 0.588 | 0.443 | 1 |
| H1d | AAGCTA | 0.04 | 0.06 | 0.212 | 0.646 | 1 |
| H1e | AGGCTA | 0.15 | 0.07 | 1.865 | 0.172 | 0.86 |

Haplotype IDs based on Pittman et al. (2005) (rs1467967, rs242557, rs3785883, rs2471738, rs9468, and rs7521, Chi-squared test (X2), significant values after adjustment for multiple testing in bold

### Example 6

### TPD is Associated with a Variation in the MAPT 3′UTR

Materials and Methods

To identify genetic variation that may be associated with TPD risk, MAPT was resequenced as described in Example 5 in a cohort of ten TPD patients and five successful cerebral aging controls. Resequencing was performed using multiplex PCR for target enrichment followed by large-scale parallel pyrosequencing of amplicons (Table 8) The target region is 140 kb in total length and completely contained within the ancestral inversion, encompassing all of MAPT, including the promoter (which overlaps with LOC100128977), introns, exons and untranslated regions, as well as the saitohin gene (STH), LOC100130148, and approximately 2 kb of KIAA1267.

Results

Using this approach, a mean base coverage of 109 reads and an average completeness of 99.5% was obtained. Sequence reads were then mapped back to the human genome project reference sequence, and 1236 variants in total were identified, 705 of which are found in dbSNP and the remaining 531 were novel.

15 variants were found to be within MAPT coding regions, 13 of which were known (rs10445337, rs1052551, rs1052553, rs11568305, rs17651549, rs17652121, rs2258689, rs62063786, rs62063787, rs62063845, rs63750072, rs63750222, rs63750417). Of these variants, 11 reside in exons that are not expressed in the central nervous system (i.e., exons 4a, 6 and 8) (Andreadis (2005)). The remaining four coding region variants in exons 7 and 9 are synonymous. Consistent with previous reports, much of the variation is derived from differences between H1 and H2, which are in complete linkage disequilibrium (Stefansson et al. (2005)).

Next, an association analysis was performed testing 20 variants identified by resequencing (2 coding and 18 noncoding). Variants were selected for validation and analysis based on their frequency, genomic location and p value. The two most significant associations (rs5820605; p=0.032) and (rs35134656; p=0.015), identified when the TPD patients (n=34) are compared to all controls (n=48) do not survive strict Bonferroni correction for multiple testing (Table 10). However, when TPD is directly compared to successful cerebral aging controls (n=28), the differences in these two variants are highly significant, with both maintaining significance following correction: rs5820605 (p=0.002, OR 0.32, CI95=0.15-0.67 and rs35134656 (p=0.002, OR 4.76, CI=1.66-13.66). The frequency of rs5820605 is 0.33 in TPD, but increases to 0.61 in successful cerebral aging, suggesting that it may be protective. In contrast, rs35134656 has a frequency of 0.09 in the controls, but increases to 0.32 in TPD, suggesting that it is a risk allele.

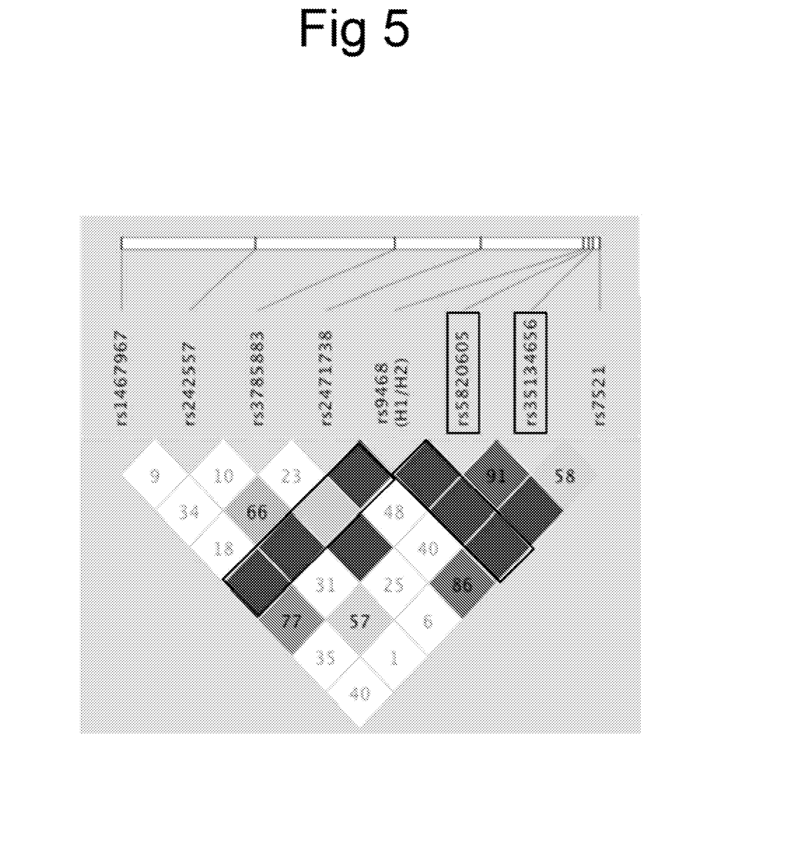

It was determined that rs5820605 and rs35134656 are in linkage disequilibrium (LD) with the H2-tagging SNP rs9486, showing that both these variants are on the H1 background (FIG. 5). In fact, both variants are situated within the MAPT 3′UTR. Both of these variants are insertion-deletion polymorphisms. The two variants are 1246 base pairs from each other, but are not in linkage disequilibrium. Instead, sequence alignment reveals two instances of a conserved 11 bp motif (CAGNCACCCCT) (SEQ ID NO: 3) containing these two polymorphisms (FIG. 6). In both cases, the TPD-associated form of the polymorphisms are predicted to disrupt this consensus, suggesting that rs5820605 and rs35134656 are contained within functional elements in the MAPT 3′UTR.

### Example 7

### Variation in the 3′UTR of MAPT Impacts Post-transcriptional Expression of Tau at Baseline and In Response to Aβ

Materials and Methods

RNA extraction was performed by disruption of fresh-frozen brain tissue by pulverization under liquid nitrogen, lysed in QIAzol and homogenized using a QlAshredder spin column (Qiagen, Valencia, Calif.). RNA was extracted using an RNeasy Mini Kit (Qiagen). cDNA synthesis was performed using a First Strand cDNA Synthesis Kit (Origene, Rockville, Md.), and used a template (1:4 dilution) in 20 μl reactions.

Primers and probes specific for 3R tau, 4R tau, total tau and GAPDH and TaqMan Gene Expression Master Mix (Applied Biosystems, Foster, Calif.) were used for quantitative real-time polymerase chain reactions on a Mastercycler ep realplex (Eppendorf, Hauppauge, N.Y.), using the following settings: 95° C. for 10 minutes followed by 40 cycles of 95° C. for 15 s and 60° C. for 1 minute. For QPCR of APP mRNA, FastStart Universal SYBR Green Master (Roche Applied Science) was used with primers described by Gilberto et al. (2008). The mRNA level was normalized to GAPDH.

The dual-luciferase 3′UTR reporter assay was performed using sequences covering the full-length MAPT 3′UTR that were PCR amplified from genomic DNA using the following primers:

Forward primer
(SEQ ID NO: 4)
5′-AATTCTAGGCGATCGCTGAGAAGCAGGGTTTGTGATCAGG-3′;
Reverse primer
(SEQ ID NO: 5)
5′-ATTTTATTGCGGCCAGCGGCCGCGGTGCGTGGGAAAGAACTTA-3′;

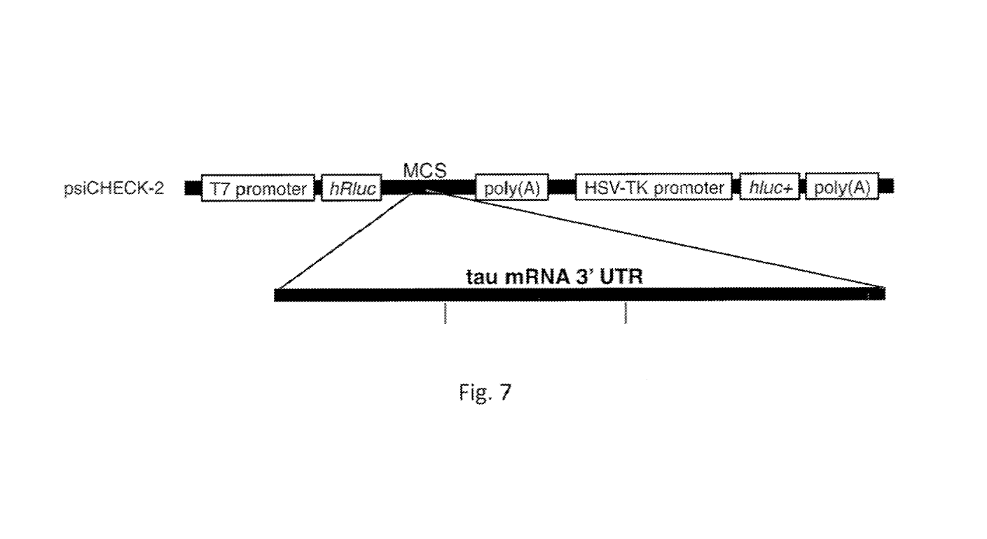

and cloned into the XhoI-NotI sites of the dual luciferase reported vector psiCHECK-2 (Promega, Madison, Wis.) using the In-Fusion HD Cloning Kit (Clontech, Mountain View, Calif.) downstream of Renilla luciferase. A full-length 3′UTR sequences of H1 amplified from human brain genomic DNA from one TPD patient harboring the rs5820605 deletion, designated H1^{3′UTR-rs5820605}, one control with the insertion, designated H1^{3′UTR}, as well as one H2, designated H2^{3′UTR} were inserted into the dual luciferase reporter system as shown in FIG. 7. Luciferase reporter constructs were transfected into SH-SY5Y cells using NeuroPORTER (Genlantis, San Diego, Calif.). Luciferase activity was assayed using a Dual-lo Luciferase Assay Kit (Promega) and expressed as the ratio of Renilla to firefly luciferase.

SH-SY5Y cells were grown either in Dulbecco's modified Eagle's medium (DMEM) or DMEM/F12 medium (Cellgro) supplemented with 10% fetal bovine serum, 2 mM glutamine, 100 units/ml penicillin, and 100 μg/ml streptomycin in a humidified atmosphere of 5% CO2 and 95% air at 37° C.

Results

The potential that there may be risk-associated variants within the 3′UTR of MAPT in TPD prompted the consideration of alterations that occur in TPD at the mRNA level. Many cis-acting elements are contained within 3′UTRs that impact virtually all levels of gene expression.

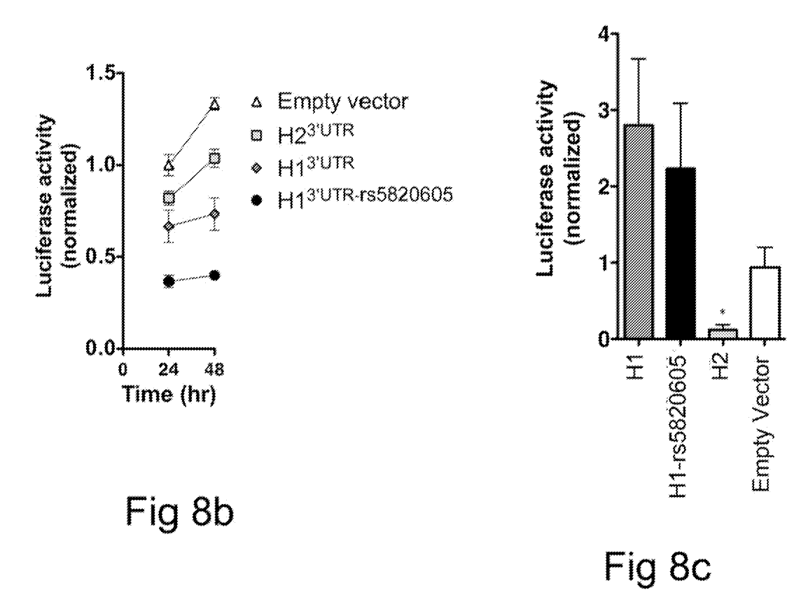

QPCR analysis did not reveal any differences in the levels or ratios of 3R and 4R tau among TPD, AD and control (FIG. 8a).

Using the reporter system, luciferase activity was compared between the three constructs described above (FIG. 8b). Following normalization, the 3′UTR constructs displayed significantly different expression levels (p=0.0004, one-way ANOVA). The H2^{3′UTR} displayed the highest signal at baseline, while surprisingly the H1^{3′UTR-rs5820605} construct has the lowest luminosity, and the control H1^{3′UTR}, was intermediate. These findings are consistent with the hypothesis that genetic variation in the MAPT 3′UTR impacts post-transcriptional regulation of tau expression.

As shown in Example 2, there has been no significant amyloid accumulation in the brains of TPD patients. However, the possibility of a transient elevation of Aβ may trigger a cascade that ultimately leads to neurofibrillary degeneration and cell death. Using reporter gene constructs, the response of the different MAPT 3′UTRs to Aβ was determined.

A dramatic decrease in luciferase expression of cells transfected with the H23′UTR construct (p=0.018, one-way ANOVA), and an increase in expression of both the H1^{3′UTR-rs5820605} construct and the control H1^{3′UTR}, was seen in response to the exogenous application of 300 nM of Aβ (FIG. 8c). There is no significant difference between the H1 constructs, suggesting that the rs5820605 polymorphism does not mediate post-transcriptional alterations in tau expression in response to Aβ.

### REFERENCES

Abraham et al. (2009) An association study of common variation at the MAPT locus with late-onset Alzheimer's disease. Am J Med Genet B Neuropsychiatr Genet 150B: 1152-1155
Albert et al. (2011) The diagnosis of mild cognitive impairment due to Alzheimer's disease: recommendations from the National Institute on Aging-Alzheimer's Association workgroups on diagnostic guidelines for Alzheimer's disease. Alzheimer's & Dementia: The Journal of the Alzheimer's Association 7: 270-279
Ambros and Horvitz (1987) The lin-14 locus of Caenorhabditis elegans controls the time of expression of specific postembryonic developmental events. Genes & Development 1: 398-414
Andreadis (2005) Tau gene alternative splicing: expression patterns, regulation and modulation of function in normal brain and neurodegenerative diseases. Biochim Biophys Acta 1739: 91-103
Aronov et al. (2001) Axonal tau mRNA localization coincides with tau protein in living neuronal cells and depends on axonal targeting signal. The Journal of Neuroscience: The Official Journal of the Society for Neuroscience 21: 6577-6587
Aronov et al. (1999) Identification of 3′UTR region implicated in tau mRNA stabilization in neuronal cells. Journal of Molecular Neuroscience 12: 131-145
Baker et al. (1999) Association of an extended haplotype in the tau gene with progressive supranuclear palsy. Hum Mol Genet 8: 711-715
Bancher and Jellinger (1994) Neurofibrillary tangle predominant form of senile dementia of Alzheimer type: a rare subtype in very old subjects. Acta Neuropathol 88: 565-570
Bekris et al. (2010) The genetics of Parkinson disease. Journal of Geriatric Psychiatry and Neurology 23: 228-242
Bouras et al. (1994) Regional distribution of neurofibrillary tangles and senile plaques in the cerebral cortex of elderly patients: a quantitative evaluation of a one-year autopsy population from a geriatric hospital. Cerebral cortex 4: 138-150
Braak and Braak (1991) Neuropathological stageing of Alzheimer-related changes. Acta Neuropathol 82: 239-259
Braak et al. (1993) Staging of Alzheimer-related cortical destruction. Eur Neurol 33: 403-408
Cairns et al. (2007) Neuropathologic diagnostic and nosologic criteria for frontotemporal lobar degeneration: consensus of the Consortium for Frontotemporal Lobar Degeneration. Acta Neuropathol 114: 5-22
Clark et al. (2009) Association of glucocerebrosidase mutations with dementia with lewy bodies. Archives of Neurology 66: 578-583
Corder et al. (1993) Gene dose of apolipoprotein E type 4 allele and the risk of Alzheimer's disease in late onset families. Science 261: 921-923
Crary et al. (2006) Atypical protein kinase C in neurodegenerative disease I: PKMzeta aggregates with limbic neurofibrillary tangles and AMPA receptors in Alzheimer disease. Journal of Neuropathology and Experimental Neurology 65: 319-326
Davidsson et al. (2001) Reduced expression of amyloid precursor protein, presenilin-1 and rab3a in cortical brain regions in Alzheimer's disease. Dement Geriatr Cogn Disord 12: 243-250
Devanand et al. (2010) Pittsburgh compound B (11C-PIB) and fluorodeoxyglucose (18 F-FDG) PET in patients with Alzheimer disease, mild cognitive impairment, and healthy controls. Journal of Geriatric Psychiatry and Neurology 23: 185-198
Dickson (2009) Neuropathology of non-Alzheimer degenerative disorders. International Journal of Clinical and Experimental Pathology 3:1-23.
Dickson et al. (1992) Identification of normal and pathological aging in prospectively studied nondemented elderly humans. Neurobiology of Aging 13: 179-189
Di Maria et al. (2000) Corticobasal degeneration shares a common genetic background with progressive supranuclear palsy. Ann Neurol 47: 374-377
Foster et al. (2012) Diagnostic Classification with Amyloid PET and FDG-PET among Clinically Diagnosed Alzheimer's Disease Patients in the Alzheimer's Disease Neuroimaging Initiative. Human Amyloid Imaging Abstract Jan 1:
Gasparini et al. (2007) Frontotemporal dementia with tau pathology. Neurodegener Dis 4: 236-253
Gavett et al. (2010) Mild traumatic brain injury: a risk factor for neurodegeneration. Alzheimers Res Ther 2: 18
Ghebranious et al. (2005) Detection of ApoE E2, E3 and E4 alleles using MALDI-TOF mass spectrometry and the homogeneous mass-extend technology. Nucleic Acids Research 33: e149
Giliberto et al. (2008) Evidence that the Amyloid beta Precursor Protein-intracellular domain lowers the stress threshold of neurons and has a “regulated” transcriptional role. Molecular Neurodegeneration 3: 12
Hardy (2006) Alzheimer's disease: the amyloid cascade hypothesis: an update and reappraisal. Journal of Alzheimer's disease 9: 151-153
Hardy and Selkoe (2002) The amyloid hypothesis of Alzheimer's disease: progress and problems on the road to therapeutics. Science 297: 353-356
Hayesmoore et al. (2009) The effect of age and the H1c MAPT haplotype on MAPT expression in human brain. Neurobiol Aging 30: 1652-1656
Herrmann et al. (2011) Current and Emerging Drug Treatment Options for Alzheimer's Disease: A Systematic Review. Drugs
Hutton et al. (1998) Association of missense and 5′-splice-site mutations in tau with the inherited dementia FTDP-17. Nature 393: 702-705.
Hyman et al. (2012) National Institute on Aging-Alzheimer's Association guidelines for the neuropathologic assessment of Alzheimer's disease. Alzheimer's & Dementia: The Journal of the Alzheimer's Association 8: 1-13
Ikeda et al. (1999) Clinical aspects of ‘senile dementia of the tangle type’—a subset of dementia in the senium separable from late-onset Alzheimer's disease. Dement Geriatr Cogn Disord 10: 6-11
Ikeda et al. (1997) Senile Dementia with Abundant Neurofibrillary Tangles Without Accompanying Senile Plaques: A Subset of Senile Dementia with High Incidence of the APOE e2 Allele. In: Iqbal K W B, Nishimura T, Takeda M, Wisniewski H M (ed) Alzheimer's Disease: Biology, Diagnosis and Therapeutics 1 edn. John Wiley & Sons, New York
Ikeda et al. (1997) A subset of senile dementia with high incidence of the apolipoprotein E epsilon2 allele. Ann Neurol 41: 693-695
Iseki et al. (2006) Immunohistochemical investigation of neurofibrillary tangles and their tau isoforms in brains of limbic neurofibrillary tangle dementia. Neurosci Lett 405: 29-33
Inner et al. (2010) Dendritic function of tau mediates amyloid-beta toxicity in Alzheimer's disease mouse models. Cell 142, 387-397
Jarrett et al. (1993) The carboxy terminus of the beta amyloid protein is critical for the seeding of amyloid formation: implications for the pathogenesis of Alzheimer's disease. Biochemistry 32: 4693-4697
Jellinger and Attems (2007) Neurofibrillary tangle-predominant dementia: comparison with classical Alzheimer disease. Acta Neuropathol 113: 107-117
Jellinger and Bancher (1998) Senile dementia with tangles (tangle predominant form of senile dementia). Brain Pathology 8: 367-376
Junn and Mouradian (2012) MicroRNAs in neurodegenerative diseases and their therapeutic potential. Pharmacol Ther 133: 142-150
Katzman et al. (1988) Clinical, pathological, and neurochemical changes in dementia: a subgroup with preserved mental status and numerous neocortical plaques. Annals of Neurology 23, 138-144
Kidd (1963) Paired helical filaments in electron microscopy of Alzheimer's disease. Nature 197: 192-193
Ksiezak-Reding and Wall (1994) Mass and physical dimensions of two distinct populations of paired helical filaments. Neurobiology of Aging 15: 11-19
Lee et al. (1993) The C. elegans heterochronic gene lin-4 encodes small RNAs with antisense complementarity to lin-14. Cell 75: 843-854
Maniatis et al. (1982) Molecular Cloning: A Laboratory Manual (Cold Spring Harbor Laboratory, Cold Spring Harbor, N.Y.
Mattson et al. (1993) Evidence for excitoprotective and intraneuronal calcium-regulating roles for secreted forms of the beta-amyloid precursor protein. Neuron 10: 243-254
McKhann et al. (2011) The diagnosis of dementia due to Alzheimer's disease: recommendations from the National Institute on Aging-Alzheimer's Association workgroups on diagnostic guidelines for Alzheimer's disease. Alzheimer's & Dementia: The Journal of the Alzheimer's Association 7, 263-269
McKhann et al. (1984) Clinical diagnosis of Alzheimer's disease: report of the NINCDS-ADRDA Work Group under the auspices of Department of Health and Human Services Task Force on Alzheimer's Disease. Neurology 34: 939-944
Mirra et al. (1991) The Consortium to Establish a Registry for Alzheimer's Disease (CERAD). Part II. Standardization of the neuropathologic assessment of Alzheimer's disease. Neurology 41: 479-486
Montine et al. (2012) National Institute on Aging-Alzheimer's Association guidelines for the neuropathologic assessment of Alzheimer's disease: a practical approach. Acta Neuropathologica 123: 1-11
Mukherjee et al. (2007) Haplotype-based association analysis of the MAPT locus in late onset Alzheimer's disease. BMC Genet 8: 3
Murray et al. (2011) Neuropathologically defined subtypes of Alzheimer's disease with distinct clinical characteristics: a retrospective study. Lancet neurology 10: 785-796
Myers et al. (2005) The H1c haplotype at the MAPT locus is associated with Alzheimer's disease. Hum Mol Genet 14: 2399-2404
Myers et al. (2007) The MAPT H1c risk haplotype is associated with increased expression of tau and especially of 4 repeat containing transcripts. Neurobiol Dis 25: 561-570
Nelson et al. (2010) Thinking outside the box; Alzheimer-type neuropathology that does not map directly onto current consensus recommendations. Journal of Neuropathology and Experimental Neurology 69, 449-454
Nelson et al. (2009) Brains with medial temporal lobe neurofibrillary tangles but no neuritic amyloid plaques are a diagnostic dilemma but may have pathogenetic aspects distinct from Alzheimer disease. J Neuropathol Exp Neurol 68: 774-784
Noda et al. (2006) Quantitative analysis of neurofibrillary pathology in a general population to reappraise neuropathological criteria for senile dementia of the neurofibrillary tangle type (tangle-only dementia): the Hisayama Study. Neuropathology 26: 508-518
Pittman et al. (2005) Linkage disequilibrium fine mapping and haplotype association analysis of the tau gene in progressive supranuclear palsy and corticobasal degeneration. J Med Genet 42: 837-846
Price and Morris (1999) Tangles and plaques in nondemented aging and “preclinical” Alzheimer's disease. Ann Neurol 45: 358-368
Purcell et al. (2007) PLINK: a tool set for whole-genome association and p opulation-based linkage analyses. American Journal of Human Genetics 81: 559-575
Rademakers et al. (2005) High-density SNP haplotyping suggests altered regulation of tau gene expression in progressive supranuclear palsy. Hum Mol Genet 14: 3281-3292
Roberson et al. (2007) Reducing endogenous tau ameliorates amyloid beta-induced deficits in an Alzheimer's disease mouse model. Science 316: 750-754
Rowe and Kahn (1987) Human aging: usual and successful. Science 237: 143-149
Rozen and Skaletsky (2000) Primer3 on the WWW for general users and for biologist programmers. Methods in molecular biology 132, 365-386
Sambrook et al. (1989) Molecular Cloning: A Laboratory Manual (Cold Spring Harbor Laboratory, 2nd Ed., Cold Spring Harbor, N.Y.
Santa-Maria et al. (2012) The MAPT H1 haplotype is associated with tangle-predominant dementia. Acta Neuropathologica 124:693-704.
Saunders et al. (1993) Association of apolipoprotein E allele epsilon 4 with late-onset familial and sporadic Alzheimer's disease. Neurology 43: 1467-1472
Savva et al. (2009) Age, neuropathology, and dementia. N Engl J Med 360: 2302-2309
Schmidt et al. (2005) Tissue processing prior to protein analysis and amyloid-beta quantitation. Methods Mol Biol 299: 267-278
Sherry et al. (2001) dbSNP: the NCBI database of genetic variation. Nucleic Acids Research 29: 308-311.
Sperling et al. (2011) Toward defining the preclinical stages of Alzheimer's disease: recommendations from the National Institute on Aging-Alzheimer's Association workgroups on diagnostic guidelines for Alzheimer's disease. Alzheimer's & Dementia: The Journal of the Alzheimer's Association 7, 280-292.
Stefansson et al. (2005) A common inversion under selection in Europeans. Nat Genet 37: 129-137
Stein et al. (2004) Neutralization of transthyretin reverses the neuroprotective effects of secreted amyloid precursor protein (APP) in APPSW mice resulting in tau phosphorylation and loss of hippocampal neurons: support for the amyloid hypothesis. J Neurosci 24: 7707-7717
Takahashi et al. (2002) Morphological and biochemical correlations of abnormal tau filaments in progressive supranuclear palsy. Journal of Neuropathology and Experimental Neurology 61: 33-45
Terry et al. (1991) Physical basis of cognitive alterations in Alzheimer's disease: synapse loss is the major correlate of cognitive impairment. Annals of Neurology 30: 572-580
Thinakaran and Koo (2008) Amyloid precursor protein trafficking, processing, and function. J Biol Chem 283: 29615-29619
Trojanowski et al. (2010) Update on the biomarker core of the Alzheimer's Disease Neuroimaging Initiative subjects. Alzheimer's & Dementia: The Journal of the Alzheimer's Association 6: 230-238
Ulrich et al. (1992) Abundant neurofibrillary tangles without senile plaques in a subset of patients with senile dementia. Neurodegeneration 1: 257-284
Vandrovcova et al. (2010) Disentangling the role of the tau gene locus in sporadic tauopathies. Current Alzheimer Research 7: 726-734
Vonsattel et al. (2008) Twenty-first century brain banking. Processing brains for research: the Columbia University methods. Acta Neuropathol 115, 509-532
Walsh and Selkow (2007) A beta oligomers—a decade of discovery. J Neurochem 101: 1172-1184
Wu et al. (2011) Decrease in brain soluble amyloid precursor protein beta (sAPPbeta) in Alzheimer's disease cortex. Journal of Neuroscience Research 89: 822-832
Yamada (2003) Senile dementia of the neurofibrillary tangle type (tangle-only dementia): neuropathological criteria and clinical guidelines for diagnosis. Neuropathology 23: 311-317
Yamada et al. (2001) Senile dementia of the neurofibrillary tangle type: a comparison with Alzheimer's disease. Dement Geriatr Cogn Disord 12: 117-126
Zuo et al. (2006) Variation at APOE and STH loci and Alzheimer's disease. Behav Brain Funct 2: 13

1.-10. (canceled)

11. A method of screening, diagnosing, predicting, or identifying tangle-predominant dementia in a subject, comprising:
a. obtaining biological tissue or bodily fluid from the subject;
b. isolating a sample of protein from the biological tissue or bodily fluid;
c. measuring the quantity of N-terminal fragments of amyloid precursor protein in the sample of protein; and
d. comparing the quantity of N-terminal fragments of amyloid precursor protein in (c) with a reference value of the quantity of N-terminal fragments of amyloid precursor protein, the reference value representing a known diagnosis or prediction normal cognitive function, and finding a deviation in the quantity of the N-terminal fragments of amyloid precursor protein measured in (c) from the reference value;
wherein a deviation in the quantity of the N-terminal fragments of amyloid precursor protein measured in (c) from the reference value of the quantity of the N-terminal fragments of amyloid precursor, determines, diagnoses, predicts or identifies the subject as having tangle-predominant dementia.

12. The method of claim 11, wherein the subject is human.

13. The method of claim 11, wherein the subject is suffering with cognitive impairment ranging from mild to severe or with pre-dementia in the prodromal phase.

14. The method of claim 11, wherein the biological tissue is brain, epidermal, blood or plasma.

15. The method of claim 11, wherein the bodily fluid is cerebrospinal fluid, saliva, plasma, sweat or urine.

16. The method of claim 11, wherein the protein is isolated and purified from the biological tissue or bodily fluid.

17. The method of claim 11, wherein the quantity of N-terminal fragments of amyloid precursor protein in the sample of protein is measured using an antibody that recognizes or binds to the N-terminal fragments of amyloid precursor protein.

18. The method of claim 11, wherein the level of N-terminal fragments of amyloid precursor protein in the sample of protein is measured by an assay selected from the group consisting of quantitative Western blots, immunoblots, quantitative mass spectrometry, enzyme-linked immunosorbent assays, radioimmunoassays, immunoradiometric assays, immunoenzymatic assays and sandwich assays.

19. The method of claim 11, wherein the N-terminal fragment of amyloid precursor protein being measured is sAPPα, and wherein if the quantity of sAPPα in the sample of protein from the subject is increased from, or higher or greater than the reference value of sAPPα, then the subject can be determined, diagnosed, predicted or identified as having tangle-predominant dementia.

20. The method of claim 11, wherein the N-terminal fragment of amyloid precursor protein being measured is sAPPβ, and wherein if the quantity of sAPPβ in the sample of protein from the subject is decreased from, or lower or less than the reference value of sAPPβ, then the subject can be determined, diagnosed, predicted or identified as having tangle-predominant dementia.

21. A kit for performing the method of claim 11, comprising (i) an antibody or antibodies that recognizes or binds to the sAPPα, and sAPPβ for measuring the quantity of the sAPPα, and sAPPβ in a sample from a subject, and (ii) a reference value of the sAPPα, and sAPPβ or a means for establishing a reference value, wherein the reference value represents a known diagnosis or prediction for tangle-predominant dementia.

22.-38. (canceled)

39. A method of screening, diagnosing, predicting or identifying tangle-predominant dementia in a subject, comprising:
a. obtaining biological tissue or bodily fluid from the subject;
b. isolating and purifying a sample of nucleic acid from the biological tissue or bodily fluid; and
c. detecting the presence of H1 haplotype of the MAPT locus comprising the nucleotide sequence of SEQ ID NO: 6 in the sample of nucleic acid;
wherein the presence of the H1 haplotype of the MAPT locus comprising the nucleotide sequence of SEQ ID NO: 6 in the sample of nucleic acid is detected by an assay selected from the group consisting of (a) hybridizing a H1 haplotype probe comprising the nucleotide sequence of SEQ ID NO: 6 to the nucleic acid sample, and detecting the presence of hybridization products, (b) hybridizing an allele-specific probe to nucleic acid sample and detecting the presence of hybridization products in the sample, (c) amplifying all or part of the MAPT locus from the nucleic acid sample to produce an amplified sequence and sequencing the amplified sequence, (d) amplifying all or part of the MAPT locus from the nucleic acid sample using primers for the H1 haplotype of the MAPT locus comprising the nucleotide sequence of SEQ ID NO: 6 and determining the presence of a hybridization product in the sample, (e) molecularly cloning all or part of the MAPT locus from the nucleic acid sample to produce a cloned sequence and sequencing the cloned sequence, (f) amplification of MAPT locus sequences in the nucleic acid sample and hybridization of the amplified sequences to nucleic acid probes which comprise the H1 haplotype of the MAPT locus comprising the nucleotide sequence of SEQ ID NO: 6 and (g) in situ hybridization of the MAPT locus of the nucleic acid sample with nucleic acid probes which comprise the H1 haplotype of the MAPT locus comprising the nucleotide sequence of SEQ ID NO: 6; and
wherein the presence of the H1 haplotype of the MAPT locus comprising the nucleotide sequence of SEQ ID NO: 6 determines, diagnoses, predicts or identifies the subject as having tangle-predominant dementia.

40. (canceled)

41. The method of claim 47, wherein the subject is human.

42. The method of claim 47, wherein the subject is suffering with cognitive impairment ranging from mild to severe or with pre-dementia in the prodromal phase.

43. The method of claim 47, wherein the biological tissue is brain, epidermal, blood or plasma.

44. The method of claim 47, wherein the bodily fluid is cerebrospinal fluid, saliva, plasma, sweat or urine.

45. The method of claim 47, wherein the nucleic acid is RNA, cDNA or genomic DNA.

46. (canceled)

47. A method of screening, diagnosing, predicting or identifying tangle-predominant dementia in a subject, comprising:
a. obtaining biological tissue or bodily fluid from the subject;
b. isolating and purifying a sample of nucleic acid from the biological tissue or bodily fluid; and
c. detecting the presence of the polymorphisms designated rs35134565 and rs5820605 in the sample of nucleic acid;
wherein the presence of the polymorphisms designated rs35134565 and rs5820605 in the sample of nucleic acid is detected by an assay selected from the group consisting of (a) hybridizing a probe to the nucleic acid sample, and detecting the presence of hybridization products, (b) hybridizing an allele-specific probe to nucleic acid sample and detecting the presence of hybridization products in the sample, (c) amplifying all or part of the 3′UTR of MAPT locus from the nucleic acid sample to produce an amplified sequence and sequencing the amplified sequence, (d) amplifying all or part of the 3′UTR from MAPT locus from the nucleic acid sample using primers for the polymorphisms designated rs35134565 and rs5820605 and determining the presence of a hybridization product in the sample, (e) molecularly cloning all or part of the 3′ UTR of MAPT locus from the nucleic acid sample to produce a cloned sequence and sequencing the cloned sequence, (f) amplification of the 3′UTR of MAPT locus sequences in the nucleic acid sample and hybridization of the amplified sequences to nucleic acid probes which comprise the polymorphisms designated rs35134565 and rs5820605 and (g) in situ hybridization of the 3′ UTR of MAPT locus of the nucleic acid sample with nucleic acid probes which comprise the polymorphisms designated rs35134565 and rs5820605; and
wherein the presence of the polymorphism designated rs35134565 determines, diagnoses, predicts or identifies the subject as having tangle-predominant dementia, and the absence of the polymorphism designated rs5820605 determines, diagnoses, predicts or identifies the subject as having tangle-predominant dementia.


SEQUENCE LISTING (8 sequences)

SEQ ID NO: 1 - DNA, 31 bp, Homo sapiens
ccgtgagcctcagccacccctcagactgggt

SEQ ID NO: 2 - DNA, 30 bp, Homo sapiens
acggccactgcagtcaccctgtctgcgccg

SEQ ID NO: 3 - DNA, 11 bp, Homo sapiens
  misc_feature (4)..(4): n is a, c, g, or t
cagncacccct

SEQ ID NO: 4 - DNA, 40 bp, Homo sapiens
aattctaggcgatcgctgagaagcagggtttgtgatcagg

SEQ ID NO: 5 - DNA, 46 bp, Homo sapiens
attttattgcggccagcggccgcggtggtgcgtgggaaagaactta

SEQ ID NO: 6 - DNA, 4433 bp, homo sapiens
accctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaagg

SEQ ID NO: 7 - DNA, 52 bp, Homo sapiens
  misc_feature (27)..(27): n is a, c, g, or t
ggctgggcgcggacatggaggacgtgngcggccgcctggtgcagtaccgcgg

SEQ ID NO: 8 - DNA, 53 bp, Homo sapiens
  misc_feature (27)..(27): n is a, c, g, or t
tccgcgatgccgatgacctgcagaagngcctggcagtgtaccagggccggggc